(19) United States
(12) Patent Application Publication
(10) Pub. No.: US 2011/0119780 A1
(43) Pub. Date: May 19, 2011

(54) SCREENING FOR NON-GENOTOXIC CARCINOGENS

(75) Inventors: Charles Roland Wolf, Dundee (GB); Clifford Roy Elcombe, Dundee (GB)

(73) Assignee: ITI SCOTLAND LIMITED, Glascow, Strathclyde (GB)

(21) Appl. No.: 12/920,717

(22) PCT Filed: Mar. 9, 2009
(86) PCT No.: PCT/GB2009/000642

§ 371 Date: Feb. 4, 2011

(30) Foreign Application Priority Data
Mar. 7, 2008 (GB) 0804310.1

(51) Int. Cl. A01K 67/00; C12Q 1/02; C12N 5/00

(52) U.S. Cl. 800/18; 435/29; 435/325; 800/13; 800/14

(57) ABSTRACT

The invention relates to a method for screening for the effects of non-genotoxic carcinogens in an animal model. The invention also relates to animal models that are suitable for use in such a method, and cell lines derived from these animals for in vitro screening purposes. More specifically, the invention relates to a transgenic rodent animal which has been humanised for the nuclear transcription factors CAR, PXR and PPARα, and in which the endogenous equivalent genes have been rendered inoperable.

The invention relates to a method for screening for the effects of non-genotoxic carcinogens in an animal model. The invention also relates to animal models that are suitable for use in such a method, and cell lines derived from these animals for in vitro screening purposes.

A significant proportion of therapeutic drug candidates fail to become marketable drugs because of adverse metabolism or toxicity discovered during clinical trials. These failures represent a very significant waste of development expenditure and consequently there is a need for new technologies that can more reliably, quickly and economically predict at the pre-clinical development stage the metabolic and toxicological characteristics of drug candidates in man. At present, most pre-clinical metabolic and toxicity testing of drug candidates relies on laboratory animals, human and/or mammalian cell lines and/or tissues in culture. However, none of these methods is completely reliable in predicting metabolism or toxicity in a human subject. Metabolic and toxicological data from animals can differ significantly from that obtained from a human subject due to species differences in the biochemical mechanisms involved. In addition, interpretation of data derived from in vitro human cell cultures or isolated human tissue studies can be problematic since such systems are not available for all organs and tissues or they fail to retain the same metabolic characteristics as they possess in vivo.

A major factor associated with the assessment of the safety of drugs and other chemical agents to which we are exposed is their capacity to induce epigenetic carcinogenesis through the induction of liver growth. The capacity of agents to act in this manner is currently tested in laboratory rats or mice; however, it has been demonstrated that these tests do not necessarily reflect the human situation.

It is known in the prior art that the metabolism, distribution and toxicity of most drugs depends on their interactions with four distinct main classes of proteins, which are phase-1 drug-metabolising enzymes, such as the cytochromes P450; phase-2 drug-metabolising enzymes, such as transferases, in particular the glucuronyl transferases, glutathione transferases, sulphonyl transferases and acetyl transferases; drug transporter proteins, such as the ATP-binding cassette proteins; and finally transcription factors, such as the pregnane X receptor (PXR) and the constitutive androstane receptor (CAR) which regulate the transcription of genes encoding proteins of the preceding classes, in particular the cytochromes P450.

There are a number of reports on mouse lines that have been humanised for particular transcription factors (see Xie et al, Nature Vol 406, 435-9, 2000; or Zhang et al, Science Vol 298, 422-4, 2002) A disadvantage of these models is that the PXR or CAR genes themselves are not regulated as they are in the human by virtue of the transgene being driven by a heterologous tissue-specific promoter (albumin promoter). Consequently, over-expression of the heterologous gene can occur, leading to the result that a normal metabolic pathway is bypassed. Moreover, the PXR and CAR transgenes are derived from a cDNA rather than a genomic clone, thus the transgenic non-human animals consequently lack the sequences necessary correctly to reproduce all the transcriptional and post-transcriptional regulation of PXR or CAR expression hence their expression is restricted to the liver and may not be of a physiological level. In addition these models do not encode for splice variants of the human gene.

Our own group has generated a mouse that is double-humanised for CAR and PXR, against a null background of endogenous expression. As yet, however, mouse lines that are humanised for more than two of these receptors have not been generated, particularly where the endogenous nuclear receptors have been deleted. To our knowledge, no one has yet suggested the utility of any of these mice to elucidate the relevance of non-genotoxic carcinogens to human safety evaluation and risk assessment, or suggested that more complex models might be generated along these lines.

### SUMMARY OF THE INVENTION

According to a first aspect of the invention, there is provided a transgenic rodent animal which has been humanised for the nuclear transcription factors CAR, PXR and PPARα, and in which the endogenous equivalent genes have been rendered inoperable. Such an animal is considered to be of great potential in screening for non-genotoxic carcinogens.

A number of transcription factors have been identified which mediate the hyperplastic and hypertrophic effects of chemical agents, which generally occur on the liver. The inventors consider that out of these, of particular importance are the nuclear receptors PPARα, CAR and PXR in the regulation of the cell cycle and growth by non-genotoxic carcinogens. This aspect of the invention is thus based on the creation of a transgenic animal that has been humanised for all three of these receptors and where the endogenous host animal genes have been concomitantly rendered inoperable.

This invention provides in one single animal model a predictive approach to assessing the potential liabilities of drugs in development and of other chemical entities, that has many advantages over models described in the prior art. The generation of animal lines according to the invention markedly increases our understanding of the factors which determine drug and chemical responses in man. These models can be applied in a number of different screening scenarios, including, for example, efficacy screening, PK/PD modelling and drug and chemical safety testing.

Carcinogens can be classified as genotoxic or non-genotoxic. Genotoxins cause irreversible genetic damage or mutations by directly (either the parent molecule or a metabolite of the parent molecule) binding to DNA; there is generally no systemic level of such compounds that is considered safe. In contrast, non-genotoxins do not directly damage DNA but act in other ways to promote growth. Such toxins are classified as either cytotoxic or non-cytotoxic (no cell necrosis caused). Examples of non-genotoxins include hormones and some organic compounds. For all these compounds, there is a threshold level of exposure that is acceptable for human contact, without risk.

Current animal models are far from ideal as screens for non-genotoxic carcinogens, because the rodent receptors that are regulated by these compounds exhibit well-defined differences in their ligand specificity between mice, other rodents and humans.

Accordingly, a compound can appear toxic in the mouse, when its equivalent effect in the human would be benign or irrelevant. For example, recent evidence supports the contention that the ligand binding domains of the murine and human CAR proteins are divergent relative to other nuclear hormone receptors, resulting in species-specific differences in xenobiotic responses (Huang et al., 2004, Molecular endocrinology 18(10):2402-2408). Results reported in this paper demonstrate that a single compound can induce opposite xenobiotic responses via orthologous receptors in rodents and humans. Similar differences exist in the hamster and guinea pig.

One example of such a drug is Phenobarbital, which is currently on the market. When tested in rats and mice, both hyperplastic and hypertrophic effects are seen within days and liver tumours are evident after around 2 years. Hyperplasia is defined as a proliferation of cells within an organ or tissue beyond that which is ordinarily seen.

Hypertrophy also involves an increase in the size of an organ or area of the tissue, but involves an increase in the size of cells, with their number staying the same.] Although epidemiological studies show that phenobarbital does not cause cancer in humans, certain regulatory authorities are reluctant to ignore the rodent carcinogenicity data when assessing the safety of other products where it is too early to demonstrate their safety by retrospective epidemiology studies. As a result, there is an understandable reluctance to develop drugs and other chemicals which show hyperplastic effects in the mouse, since it is not possible to test accurately for hyperplasia in response to a drug without actually administering the drug to humans. This is clearly unacceptable unless the drug is known to be safe.

Another example is the lipid-lowering fibrate drugs, which function as agonists for the nuclear receptor peroxisome proliferator-activated receptor α (PPARα). Sustained activation of PPARα leads to the development of liver tumours in rats and mice (Cattley et al, 1998, 2004). However, humans appear to be resistant to the induction of peroxisome proliferation and the development of liver cancer in response to fibrate drugs.

A further example is provided by the family of peroxisome proliferator activated receptors (PPARs), to which various drugs were in the past developed as hypolipidic agents. The development of these drugs was stopped, as they were identified in mouse and frequently rat models to be non-genotoxic (also called epigenetic) carcinogens. It was initially thought that these differences were due to differences in expression level. However, it turns out that, for unknown reasons the human receptor upon ligand binding does not activate the cell proliferation machinery in the same way as the mouse receptor does.

In vitro screens often use human cells in an attempt to overcome the problems with cross-species variation. However, in vitro systems can only ever incorporate a small part of the drug metabolism landscape, and do not present a holistic view. Accordingly, the real in vivo effects may be disguised. For example, consider the frequent situation that arises when drugs look hazardous in vitro because a toxic by-product is generated but not in vivo because the drug activates a secondary enzyme that metabolises away the toxic by-product. It is also true that almost any compound will interact with a particular target at some level—the question of importance for drug safety is whether this interaction is physiologically relevant at the concentrations to which tissues will be exposed. The limitations inherent in the in vitro scenario make this solution inappropriate.

It would be of great utility if it were possible to demonstrate that a hyperplastic response does not occur in humans in response to drug exposure. The inventors have concluded that one effective way to generate a faithful test for safety of non-genotoxic carcinogens is through the use of rodents that have been humanised for the transcription factors with which non-genotoxic carcinogens principally interact. At the same time, it is essential to annul the expression of the equivalent endogenous rodent transcription factor genes in order to ensure that interference from non-human metabolic pathways on the functions of introduced human proteins is significantly reduced.

The inventors have noted that in general, compounds that are non-genotoxic carcinogens and cause liver tumours in rodents are PXR, CAR and PPARα ligands. These cause hyperplasia, by either or both stimulating cell proliferation and inhibiting apoptosis. Additionally, they cause hypertrophy, stimulating organelle (eg. smooth endoplasmic reticulum, peroxisomes) proliferation through the smooth endoplasmic reticulum, and enzyme induction. The barbiturates induce primarily the P450 enzyme CYP2B; steroids primarily induce CYP3A, and peroxisome proliferators primarily induce CYP4A. Some chemicals interact substantially with multiple receptors and induce multiple cytochromes P450. FIG. 1 is the inventors' depiction of how drug compounds interact with nuclear receptors and the ensuing metabolic pathways involved in drug metabolism, cell cycle regulation and growth.

Mice that have been individually humanised for CAR, PXR or PPARα currently exist. Indeed, Cheung et al 2004 monitored various physiological effects including the increase in liver body weight on exposure to drug in wild type and PPARα knockout mice, and compare this response to that seen in humanised mice. The humanised mice showed a lesser increase in liver body weight and a lack of increased replicative DNA synthesis (a marker for hyperplasia).

However, the multiply humanised models of the present invention provide a significant advantage over the deletion of individual transcription factors because of the functional redundancy between members of the same gene families.

The inventors consider that there are a number of reasons why an animal that has been humanised for all three receptors simultaneously will provide a significant improvement over the application of the current singly humanised models.

In the first instance, the transcription factors that principally mediate non-genotoxic carcinogens-regulated hyperplasia are all humanised, so resolving the problems associated with the differences in ligand specificity noted above. Therefore, one advantage of a humanised model is that it negates the issues of ligand specificity. All of PPARα, CAR, and PXR interact with exogenous ligands that transactivate gene expression, and thereby mediate pathways that the inventors consider to be potentially deleterious in the metabolism of non-genotoxic carcinogens.

The ratio of protein levels that are generated by a particular drug are also of significant importance. For example, the action of mouse PXR stimulates expression of different proteins than the action of human PXR and at different levels. The levels of a particular drug and its metabolites depends crucially on which drug metabolising enzymes and transporters are expressed and so, again, the inventors consider that it is of utmost importance for human transcription factors to be used rather than endogenous transcription factors from the test animal.

The use of the specified human transcription factors is also important from a toxicological standpoint. For example, PXR is naturally regulated by bile acids and other physiological compounds and toxic conditions such as biliary necrosis and biliary cholestasis can result from exposure to a particular drug. It may therefore be that as a result of differences between drug metabolism between human and a test animal, a toxic effect will be noted in that animal that would not be evident in the human.

One major advantage of these triple humanised animals on a triple null background is that there is significant redundancy between these transcription factors in their response to chemicals, as one chemical agent may interact with multiple receptors. Therefore, a mouse humanised for just one of these receptors would not give the correct magnitude of response.

Furthermore, there are ways in which cross-talk may occur between different nuclear receptors that are implicated in the metabolism of non-genotoxic carcinogens. The first is cross-talk between receptors at a molecular level. The second is cross-talk at a metabolic interface, for example through generation of cross-reacting secondary metabolites, or from changes in drug disposition. Thirdly, the nuclear receptors themselves can cross-talk and modulate each others' levels of expression and functions. A particular level of drug may activate genes that transactivate other genes, so leading to further levels of complication.

Therefore, only by having a complex panel of humanised receptors will it give the bona fide response that is anticipated in man. Such cross-talk might in principle be predicted at some qualitative level, but because the magnitude of the effects and the extent of any feedback mechanisms are inherently unpredictable, this undermines the value of any system that does not incorporate all the necessary elements of the system at physiologically relevant levels.

One example where cross-talk between these receptors may be of key importance in defining the eventual outcome is the fact that CAR, PXR and PPARα transcription factors, in addition to their activation by exogenous ligands, are regulated by perturbations in fatty acid homeostasis. It will be advantageous, therefore, to have mice that are humanised for all these receptors simultaneously.

Also, the simultaneous humanisation of animals at all three gene loci dramatically reduces the number of animals required to establish clearly whether a chemical agent has the capacity to induce hyperplasia, as it negates the need for carrying out multiple experiments on individual humanised animals.

The inventors have noted that the capacity of promoters to induce enzyme expression is different in different tissues. This adds significant weight to the contention that human transcription factors should be used rather than the endogenous transcription factors from the host animal. Accordingly, the regulatory sequences of the transcription factors and the genes that they regulate should mirror the natural physiological condition as closely as possible.

Animals according to the invention may be any non-human species, for example a rodent, for instance a rat, hamster or a guinea pig, or another species such as a monkey, pig, rabbit, or a canine or feline, or an ungulate species such as ovine, caprine, equine, bovine, or a non-mammalian animal species. More preferably, the transgenic non-human animal or mammal and tissues or cells are derived from a rodent, more preferably, a mouse.

Although the use of transgenic animals poses questions of an ethical nature, the benefit to man from studies of the types described herein is considered vastly to outweigh any suffering that might be imposed in the creation and testing of transgenic animals. As will be evident to those of skill in the art, drug therapies require animal testing before clinical trials can commence in humans and under current regulations and with currently available model systems, animal testing cannot be dispensed with. Any new drug must be tested on at least two different species of live mammal, one of which must be a large non-rodent. Experts consider that new classes of drugs now in development that act in very specific ways in the body may lead to more animals being used in future years, and to the use of more primates. For example, as science seeks to tackle the neurological diseases afflicting a ‘greying population’, it is considered that we will need a steady supply of monkeys on which to test the safety and effectiveness of the next-generation pills. Accordingly, the benefit to man from transgenic models such as those described herein is not in any limited to mice, or to rodents generally, but encompasses other mammals including primates. The specific way in which these novel drugs will work means that primates may be the only animals suitable for experimentation because their brain architecture is very similar to our own.

The invention aims to reduce the extent of attrition in drug discovery. Whenever a drug fails at a late stage in testing, all of the animal experiments will in a sense have been wasted. Stopping drugs failing therefore saves test animals' lives. Therefore, although the present invention relates to transgenic animals, the use of such animals should reduce the number of animals that must be used in drug testing programmes.

The regulatory sequences governing expression of the transcription factor(s) may preferably be either of human origin, or may originate from the target animal species e.g. the mouse. Regulation of the expression of introduced human proteins should be retained such that patterns of expression in the human are reproduced.

A further advantage of the invention, particularly where the human gene is introduced into the endogenous gene locus, is that the predicted pattern of gene expression is retained. Conventionally, most workers in this field have integrated a target gene into the host genome randomly, for example using BAC transgenesis. This strategy has the limitation that it does not simultaneously introduce any enhancer elements that may lie large distances up- or down-stream of the replacement gene and so influence its pattern of gene expression. Furthermore, position effects on the expression of the transgene are frequently observed. As a result, non-bona fide expression of the introduced factor will result and, as a consequence, lead to erroneous results in any experiments using that animal.

This also represents an advantage of knocking the transcription factors into the endogenous gene locus where the effects of downstream enhancers will still be manifest.

By “endogenous equivalent gene” of the animal is intended to include any gene or gene cluster that is functionally capable of replacing the function which is rendered inoperable, i.e. any gene or genes whose expression product retains the same, similar or identical function as the human counterpart gene.

For example, the human transcription factor gene known as PXR (NR1I2 nuclear receptor subfamily 1, group I, member 2), Entrez GeneID: 8856, has a murine counterpart of the same name whose Entrez GeneID is 18171. The proteins encoded by these genes have an equivalent function in the organisms from which they are derived. Accordingly, examples include acknowledged orthologous counterparts in other organisms. The rat orthologue has Entrez GeneID 84385.

The human transcription factor referred to herein as CAR is also known as NR1I3 (nuclear receptor subfamily 1, group I, member 3) and has Entrez GeneID 9970. The rat gene is known as Nr1i3 and has Entrez GeneID 65035. The mouse gene is also known as Nr1i3 and has Entrez GeneID 12355.

The human transcription factor referred to herein as PPARα has Entrez GeneID 5465. The mouse orthologue has Entrez GeneID 19013. The rat orthologue has Entrez GeneID 25747.

The model of the invention may preferably also be humanised for the nuclear receptor AhR. Similarly, the endogenous equivalent gene should be rendered inoperable. According to one embodiment of this aspect of the invention, there is therefore provided a transgenic rodent animal which has been humanised for at least the nuclear transcription factors CAR, PXR and AhR, and in which the endogenous equivalent genes have been rendered inoperable. Such an animal is considered to be of great potential in screening for non-genotoxic carcinogens. Evidence for the creation of an animal according to this embodiment of the invention is described herein. Such an animal may also be transgenic for PPARα.

AhR is a PAS domain containing protein with a different structure to the three other nuclear transcription factors mentioned above, and which has limited or no sequence homology with nuclear receptors. There are big species differences in ligand responsiveness; even within the mouse there are polymorphisms that lead to a difference in phenotypes. The AhR protein induces the activation of enzymes that have an activation potential to convert promutagens into mutagens. For example, humans are much less sensitive to dioxins as a result of their AhR transcription factor. As a hard and fast rule, pharmaceutical companies steer well clear of any compounds that interact with AhR in order to avoid any apparent toxicity evident when looking for toxicity.

AhR is also involved in cross-talk, in a similar way to the other nuclear receptors described above. For example, interactions are evident between the receptors AhR and NRF2. Ideally, a system should incorporate both these elements under appropriate conditions.

The human transcription factor referred to herein as AhR has Entrez GeneID 196. The mouse orthologue has Entrez GeneID 11622. The rat orthologue has Entrez GeneID 25690.

HNF 1 and HNF4 are other examples of transcription factors for which the animals of the present invention may be humanised, and for which the endogenous gene may be knocked out.

Generally, examples of human and murine orthologues of nuclear transcription factors are known to those of skill in the art.

Generally, the introduced transcription factor gene will share a degree of homology with the endogenous gene with which it is equivalent. Preferably, the degree of homology will be greater than 30%, greater than 40%, greater than 50%, greater than 60%, greater than 70%, greater than 80%, greater than 90%, or even greater than 95%.

The present invention attempts to mirror the in vivo situation by providing the replacement gene in its entirety where this is possible. This means that the intron-exon junctions are retained as in the natural system so that splicing events can happen exactly as in the natural situation. Where, perhaps because of the length of a gene, it is not simple to transpose the entire gene into a transgenic system, the invention seeks to use a combination of cDNA and genomic DNA in its constructs so that important intron-exon boundaries, where the majority of splicing events occur, are retained.

According to the invention, therefore, where it is known that the majority of splice variants occur as a result of splicing variation within a particular intron, this intron is preferably incorporated as genomic DNA in the construct, while less influential intronic sequences are not retained. This has the result that levels of functional mRNA and functional protein mirror the levels that are found in vivo in response to exposure to a particular drug or drug cocktail. This is what is ideally required for a physiologically-relevant model.

Accordingly, whilst cDNA sequences may be used, in preference to these sequences, the invention may use a combination of cDNA and genomic sequences from the gene that is to be humanised. For example, in the case of a transgenic animal expressing the human PXR gene, due to the large size of more than 35 kb of the human PXR gene, the intron-exon structure between exons 4 and 6 is preferably maintained (see WO2006/064197), since most splice variants are observed in this genomic region, since it is located within the ligand-binding domain. This advantageously retains the sequence where most splice variants are observed and is conveniently located within the ligand-binding domain. Similarly, for PPARα, the encoding DNA sequence comprises at least part of intron 5 and intron 6 of the human PPARα gene (see FIG. 4). Homozygous mice humanised for PPARα have been created, and are described herein. It is found that in addition to the wild type predicted coding sequence, two splice variants (termed SV1 and SV2) are generated. Variant SV1 has been published and is a human specific alternatively spliced variant (Gervois et al, 1999). Variant SV2 is a new type of transcript with the addition of a 4 bp (GTAG) out-of-frame insert at the 3′ end of exon 5, resulting in a premature stop codon. The potential functions of truncated PPARα protein in humanised mice are not known.

Complete genomic DNA sequences will preferably be used. For instance, in the case of a transgenic animal expressing the human CAR gene, the relatively small size of the human CAR, which comprises roughly 7 kb from exon 2-9, makes it simple to retain the complete genomic structure in the targeting vector. The construct should preferably retain the intron-exon structure between exons 2 and 9 (see WO2006/064197). This advantageously retains the complete genomic structure within the targeting vector and permits coverage of all splice variants of human CAR. Preferably, the genomic human CAR sequence is fused to the translational start site of the mouse CAR gene. The human CAR sequence then contains all genomic sequences of exons 1-9. The 5′ and 3′UTRs may be human or may be retained from the mouse genome. All other parts of the coding sequences of the mouse CAR gene can be deleted.

The inventors consider it to be of utmost importance when screening for non-genotoxic carcinogens that as many as possible of the endogenous genes encoding proteins with relevant functions are rendered inoperable in the test animal. One reason for this is that there is a high degree of redundancy between drug metabolism genes such as transcription factors of the type with which the invention is concerned.

By the term “rendered inoperable” is meant that the genes or gene functions are annulled or deleted. This term is thus intended to include silencing or deletion or rendering inactive so that the host animal's endogenous equivalent gene is unable to express the gene product(s), at least not to any level that is significant to the drug metabolism process. For instance, the expression level of an annulled gene may be less than 20%, preferably less than 10%, more preferably less than 5%, more preferably less than 2%, even more preferably 1% or less of the wild type expression level. The expression of a gene rendered inoperable may preferably be decreased to the point at which it cannot be detected.

Preferably, the endogenous transcription factor functionality is deleted in all tissues of the animals of the present invention. It is a widely-accepted misconception that the liver is the only truly important tissue for drug metabolism. The reality is far from this; in fact transcription factor function is expressed across a wide range of tissues other than the liver; particular examples include the gastro-intestinal tract and the blood brain barrier. The complete abrogation of function across all tissues is thus necessary in order that the effects of the endogenous gene knockouts are manifest.

The models of the invention thus provide advantages over many other models where a particular transcription factor system has been inactivated, for example using conditional knock-out in the liver. In contrast, in the models of the present invention, deletion of the transcription factors preferably occurs in all tissues.

One example of a very good reason for the utility of the animal models of the present invention is that mice, as conventionally used, metabolise most drugs far more quickly than humans, almost by a factor of 10, generally because of slower rates of P450 metabolism. Accordingly, mice that activate such enzymes will perhaps exhibit a 30 minute half-life where the equivalent drug will have a half-life of some hours in the human. Of course, this has the effect that the normal pathways of disposition are masked in the mouse, because there is no opportunity for these to take effect. Accordingly, deleting the dominant transcription factors that govern non-genotoxic drug metabolism has the effect of removing these pathways of drug disposition from contention. Humanisation of such knockout mice for the equivalent human transcription factors then allows human drug metabolism pathways to be evaluated without interference from the endogenous mouse enzymes.

Examples of methods for rendering endogenous equivalent genes inoperable in the target animal are set out in WO2006/064197. In brief, the endogenous host gene(s) may be rendered inoperable by a number of different means, as will be clear to those of skill in the art. For example, this may be by complete deletion of the coding sequences of the genes from the host animal genome. Alternatively, deletion may be accomplished by mutation of the coding sequence, either by way of insertion, deletion or substitution of other sequences. For example, one or more mutations (such as frameshift mutations) may be generated such that any resulting RNA transcript codes for a non-functional or truncated protein. In an alternative, an insertion may be made into the chromosomal sequence to disrupt the amino acid code.

Similarly, a sequence may be exchanged with the transcription factor sequence that is being deleted, such as a selection or marker sequence that can be used as the basis for screening for successful deletants. One such strategy has been devised by Wallace et al (Cell 128, 197-209 2007), in the context of gene exchange, and this is applicable to the method of the present invention. This method envisages an exchange of sequence between mouse chromosome and a BAC or YAC vector, such that two intermolecular homologous recombination events are required for the vector-based replacement sequence to replace the endogenous genomic murine sequence.

In one preferred system, a mechanism of homologous recombination is used to exchange a gene for an alternative sequence in which the gene is not present. Such a method preferably comprises the steps of: a) incorporating a pair of site-specific recombination sites into the host animal chromosome by homologous recombination such that the target gene that is to be replaced is flanked on each side by a recombination site; and b) effecting recombination between the site-specific recombination sites such that the target gene is excised from the chromosome, replaced by a residual site-specific recombination site.

Methods for performing homologous recombination are known in the art and exploit regions of homology between exogenously supplied DNA molecules and the target chromosome to introduce the RT sites. Under this methodology, 5′ and 3′ homology arms in the replacement sequence drive recombination between the replacement sequence and target such that the gene is deleted. A methodology utilising this strategy is reported in the applicant's co-pending patent application no. GB0718029.2 filed on 14 Sep. 2007 entitled “Two step cluster deletion and humanisation” and PCT/GB2008/003084, of the same title. This is perfectly applicable to the method of the present invention.

In this arrangement, each of the replacement sequences is designed such that between the 5′ and 3′ homology arms lies a selection marker and at least one recombinase target (RT) site such as loxP, lox5171, FRT or F3. In this manner, it is possible to select for successful incorporation of both replacement nucleic acids that would thus flank the gene to be deleted. One of the flanking sequences can be designed so that a replacement human transcription factor gene sequence lies outside the RT site. It is then technically simple to excise the gene by exposure of the cells to an appropriate site-specific recombinase (SSR) that recognises the RT sites. This effects total deletion of the host animal transcription factor gene while at the same time replacing the gene with the human equivalent. These described recombination steps are preferably performed in an embryonic stem cell, according to methods well known in the art.

A preferred strategy for generation of a transgenic line initially involves the creation of altered embryonic stem cells. The altered embryonic stem cell may be subsequently inserted into a blastocyst. Conventionally, blastocysts are isolated from a female mouse about 3 days after it has mated. Up to 20 altered embryonic stem cells may be simultaneously inserted into such a blastocyst, preferably about 16. Through insertion of altered embryonic stem cells into the blastocyst, the embryonic stem cell will become incorporated into the developing early embryo, preferably by its transplantation into a pseudo-pregnant animal, such as a mouse, which has been induced so as to mirror the characteristics of a pregnant animal. According to this methodology, the blastocyst, containing the altered embryonic stem cell, will implant into the uterine wall of the pseudo-pregnant animal and will continue to develop within that animal until gestation is. complete. The altered embryonic stem cell will proliferate and divide so as to populate all tissues of the developing transgenic animal, including its germ-line.

In one aspect of the methodology, the created transgenic animal may be a chimera, containing altered and non-altered cells within each somatic tissue and within the germ-line.

The methodology for mediating Cre/lox-mediated deletions, suitable for deleting of large fragments of DNA (200 kb to several megabases), has been described in the following papers (Li Z W, Stark G, Gotz J, Rulicke T, Gschwind M, Huber G, Muller U, Weissmann C. Generation of mice with a 200-kb amyloid precursor protein gene deletion by Cre recombinase-mediated site-specific recombination in embryonic stem cells Proc Natl Acad Sci USA. 1996 Jun. 11; 93(12):6158-62. Erratum in: Proc Natl Acad Sci USA 1996 Oct. 15; 93(21):12052; in Su H, Wang X, Bradley A. Nested chromosomal deletions induced with retroviral vectors in mice. Nat Genet. 2000 January; 24(1):92-5); Call L M, Moore C S, Stetten G, Gearhart J D; A cre-lox recombination system for the targeted integration of circular yeast artificial chromosomes into embryonic stem cells. Hum Mol Genet. 2000 Jul. 22; 9(12):1745-51).

Ultimately, two heterozygous animals produced according to the methodology above may be crossed to produce a transgenic animal that is homozygous for the human allele of the gene or genes of interest. Crossing two heterozygous transgenic animals will produce a proportion of progeny that are homozygous for the deletion.

In a further embodiment of the invention the transgenic non-human animal is produced de novo so as to include all of the aforementioned features, by the methods as hereinafter disclosed.

In another embodiment of the invention the mouse of the present invention is produced by crossing. For example, a partial deletant in which a proportion of the genes of a particular cluster have been deleted could be crossed with another partial deletant to generate animals which are deleted for all gene functions within a particular cluster.

Transgenic mice for human CAR have been created and are described in the examples included in WO2006/064197 and WO2008/149080. Detailed investigations of the induction of drug metabolism pathways in CAR humanised and knock-out mice have been performed. Various different experimental approaches have confirmed that non-human transgenic animals that are humanised with respect to CAR, or which do not express any CAR (knock-out), can readily be obtained using the methods and strategies described herein.

Transgenic mice for human PXR have also been created and again are described in the examples included in WO2006/064197 and WO2008/149080. Human PXR is found to be expressed in both the liver and GI tract of mice in the predicted manner at levels equivalent to those of the endogenous mouse gene. In this way, typical problems faced by conventional techniques of this type, such as over- or under-expression are avoided. In this model, the PXR protein has also been shown to be functional as the mice are responsive to compounds such as rifampicin and dexamethasone that are known to induce gene expression via this pathway. Strain differences between wild type and the humanised mice have been demonstrated. For example, the humanised mice are shown to be more responsive to compounds such as rifampicin, that are known to be more active to hPXR. Humanised PXR animals thus demonstrated an altered sensitivity to rifampicin relative to the wild type.

Furthermore, there was clearly greater background P450 enzyme activity as measured by 16-beta-hydroxylation of testosterone and 7-benzyloxyquinoline debenzylation between wild type and humanised PXR mice.

Transgenic animals (such as mice) and cells according to the invention preferably demonstrate the functional properties described above and in the examples herein. For example, such cells and animals preferably do not display induction of Cyp2b10 activity in response to rifampicin. However, such cells and animals do display an induction effect for Cyp3a11, not only with rifampicin but also for TCPOBOP.

Mice transgenic for both human PXR and human CAR have also been created and are described in the examples included herein. Preliminary studies have been performed on the activity of these transcription factors in combination, determined by measuring barbiturate-induced sleeping time. Sleeping time has been known for many years to be directly proportional to the hepatic cytochrome P450 activity and this activity can be at least in part ascribed to the P450 levels in the liver determined by CAR and PXR function. Whereas wild type mice given a narcotic dose of pentobarbitone slept for 21 minutes, the double humanised mice for CAR and PXR slept for 34 minutes. These mice therefore demonstrate a significant difference to their wild type controls indicating that the double humanised mouse has a marked difference in its response to drugs relative to the wild type animals.

Detailed investigations of the induction of drug metabolism pathways in PXR and CAR double-humanised and double-knock-out mice have been performed. Various different experimental approaches have confirmed that non-human transgenic animals that are humanised with respect to both PXR and CAR, or which do not express any PXR or CAR (double-knock-out), can readily be obtained using the methods and strategies described herein.

Given the results described herein, there is no technical barrier preventing the generation of animals that are also transgenic for PPARα. The relevant constructs have already been made and are described herein. Targeting strategies suitable for knock-in (humanisation) and knock-out of PPARα and the Ah receptor are described in more detail herein (see FIGS. 4 and 5). At the time of writing, Mice homozygous for CAR and PPARα and additionally heterozygous for PXR have been made and triple homozygous mice will be available shortly. Additionally, mice homozygous for humanised PXR, CAR and AhR are already available and described in the examples herein.

Ultimately, the animal models of the invention may be exploited as a background for introducing human genes that may substitute the functions of rodent enzymes, either by integrating these genes directly into the same chromosomal region (and thus replacement of the endogenous gene(s)) or through integration at alternative sites. Preferably, human genes will be integrated into the same chromosomal region, since the integrity of the chromosome will be retained and thus physiological patterns of expression and tissue distribution are likely to be similarly retained.

Accordingly, in embodiments of the invention relating to the preparation of cells and animals as previously described, such cells and animals may be subjected to further transgenesis, in which the transgenesis is the introduction of an additional gene or genes or protein-encoding nucleic acid sequence or sequences. The transgenesis may be transient or stable transfection of a cell or a cell line, an episomal expression system in a cell or a cell line, or preparation of a transgenic non-human animal by pronuclear microinjection, through recombination events in non-embryonic stem (ES) cells, random transgenesis in non-human embryonic stems (ES) cells or by transfection of a cell whose nucleus is to be used as a donor nucleus in a nuclear transfer cloning procedure.

In particular, it is envisaged that an animal according to the invention may be humanised for one or more human genes, including drug transporters, transcription factor or phase I drug metabolism enzymes, phase II drug metabolism enzymes, and so on.

For example, such an animal may be humanised for phase-1 drug metabolising enzyme i.e. a P450 enzyme. Preferred examples of P450 enzymes include one, two, three or more of CYP3A4, CYP3A5, CYP2C9, CYP2C19, CYP2D6, CYP1A1, CYP1A2, CYP2C8 and CYP2B6. The animal may be humanised for an entire gene cluster, such as the CYP3A cluster, the CYP2D cluster and/or the CYP2C cluster.

An animal according to the invention may also be humanised for a phase-2 drug-metabolising enzyme. Examples of such enzymes include the glucuronyl transferases, for instance, the UGT1A gene or gene cluster, the glutathione transferases, for instance GST (glutathione S-transferases) (including GST-ml and/or t1 clusters), the sulphonyl transferases and the acetyl transferases.

It is also preferred that the endogenous equivalent murine genes have been annulled, as set out in WO2006/064197. In the case of drug-metabolising enzyme genes, equivalence between genes can be assessed by a combination of substrate specificity, mode of regulation (for example, by transcription factors or exogenous drugs), sequence homology and tissue distribution. Certain genes have exact equivalents; examples of such genes are CYP2E1, CYP1A1 and CYP1A2. CYP2B6 and CYP2D are examples where there is only one gene in the human, but numerous equivalent genes in the mouse. There are four CYP2C genes in the human, and numerous equivalent genes in the mouse. In such circumstances, preferably at least one, more preferably two, three, four, five or more or even all of the equivalent murine genes are annulled. CYP3A4 is an example where there is no obvious orthologue in the mouse, but Cyp3a11 could be considered at least one equivalent mouse gene because of its hepatic expression, mode of regulation and sequence homology.

An animal according to the invention may also be humanised for a drug transporter protein, examples of which include the multi-drug resistance proteins, for instance mdr1 and mdr3 and multi-drug resistance-associated proteins (MRPs), for example, MRP1 and/or MRP2 and/or MRP6 or from the organic anion transporting polypeptides (OATPs). It is also preferred that the endogenous equivalent murine genes have been annulled.

Another aspect of the invention relates to cells, modified so as to possess properties according to any one of the above-described aspects of the invention. Hepatocytes and neuronal cells are preferred cell types according to the present invention. The cells may be rodent cells, in particular, mouse cells.

Cells according to this aspect of the invention may be created from transgenic mice according to the invention using standard techniques, as will be clear to the skilled reader, imbued with knowledge of the present invention. Suitable methods are described in many standard laboratory manuals, such as Davis et al., Basic Methods in Molecular Biology (1986); Sambrook Molecular Cloning; A Laboratory Manual, Third Edition (2000); Ausubel et al., 1991 [supra]; Spector, Goldman & Leinwald, 1998).

One preferred method of generating such cells is to cross a humanised mouse, as described above, with SV40 immortalised mouse (for example, the immorta-mouse; Taconic). Cells may subsequently be isolated from such animals according to well known techniques in the art. In contrast to prior art transgenic systems, which used the albumin promoter that is only active in the liver and thus only able to generate hepatocytes, cells from transgenic animals generated according to the present invention may be of a diverse selection of different cell types, including cells of significant importance to pharmacokinetics analyses, such as hepatocytes and neuronal cells.

Stem cells isolated from transgenic animals according to the invention, with properties as described above are also useful aspects of the present invention. Such cells may be pluripotent, or partially differentiated. Stem cells may be adult stem cells or embryonic stem cells. More generally, stem cells employed may be from a post-embryonic developmental stage e.g. foetal, neonatal, juvenile, or adult. Stem cells isolated in this manner may be used to generate specific types of cells such as hepatocytes and neuronal cells. Such cells also form an aspect of the present invention.

Cells or animals produced by the method of the invention can be used as model systems for determining the metabolism of drugs or other xenobiotic compounds in other organisms, particularly the human.

In a still further aspect of the invention, an assay according to the invention involves a method for screening a non-genotoxic carcinogen for safety in humans comprising exposing a non-human animal to the non-genotoxic carcinogen and monitoring for a physiological effect, wherein the animal is humanised for at least two nuclear transcription factors selected from the group consisting of PXR, CAR and PPARα, or the group consisting of PXR, CAR, AhR and PPARα, and wherein the endogenous equivalent genes have been rendered inoperable. The animal may be thus be humanised for PXR and CAR; PXR and PPARα; CAR and PPARα; PXR and AHR; PPARα and AHR; CAR and AHR or three or all four of these nuclear receptors (for example, PXR, CAR and PPARα, or PXR, CAR and AhR), in accordance with the terms of the invention set out in detail above.

Assays according to the invention may thus be practised on a wider range of animals than just the animals discussed above. As far as we are aware, prior to this disclosure, the concept of using mice that are even doubly humanised for nuclear transcription factors has not been suggested to screen non-genotoxic carcinogens for safe use in humans. Such double-humanised models are advantageous over models that only incorporate a single gene (either PXR or CAR) because many drug metabolising enzymes or drug transporters possess elements that are responsive to the binding of both CAR and PXR. Furthermore, the numbers of PXR-responsive elements often differ from the numbers of CAR-responsive elements and so regulation by both transcription factors is generally important. Consequently, models that take account of the effects of both factors are preferable and more closely mirror the physiological situation in vivo. Mice transgenic for both human PXR and human CAR have been created and are described in the examples included herein. Various different experimental approaches have confirmed that non-human transgenic animals that are humanised with respect to both PXR and CAR, or which do not express any PXR or CAR (double-knock-out), can readily be obtained using the methods and strategies described herein. Preferably, the mouse models are homozygous for the humanised genes.

The animals, tissues and cells of the present invention may be used to determine how a drug compound is metabolised. The generation of animal lines according to the aspects of the invention described above will markedly increase our understanding of the factors which determine drug and chemical responses in man and the relevance of these genes for chemical toxicity. These models can be applied to efficacy screening, PK/PD modelling and drug safety testing.

It is possible to measure a phenotypic change in the animal, such as a physiological effect. Such a physiological effect may be, for example, a disease condition (such as biliary necrosis) or a toxic side-effect. Preferred phenotypic changes include hyperplasia, hepatomegaly, P450 induction and/or hepatocellular proliferation.

It is possible to examine the rate of metabolism of a drug compound. The rate of metabolism may be determined by measuring the toxicity or activity mediated by the administration of the compound, measuring the half-life of the compound, or measuring the level of a transcription factor or drug metabolising enzyme. For example, the rate of metabolism of the compound may be measured as the rate of formation of the oxidized product or the formation of a subsequent product generated from the oxidized intermediate. Alternatively, the rate of metabolism may be represented as the half-life or rate of disappearance of the initial compound or as the change in toxicity or activity of the initial compound or a metabolite generated from the initial compound. The half-life may be measured by determining the amount of the drug compound present in samples taken at various time points. The amount of the drug compound may be quantified using standard methods such as high-performance liquid chromatography, mass spectrometry, western blot analysis using compound specific antibodies, or any other appropriate method.

It is also possible to examine whether under particular circumstances a drug compound is metabolised to a toxic or carcinogenic metabolite, for example, by measuring its covalent binding to tissues, proteins or DNA or by measuring glutathione depletion.

Preferably, measurements of the type described above are performed at more than 1, 3, 5, 10 or more time points after administration of the drug compound.

Accordingly, further aspects of the invention relate to screening methods that are provided to determine the effect of a drug compound on the activity or expression level of a transcription factor, a drug metabolising enzyme or a drug transporter protein. Such methods involve administering a drug compound to a transgenic animal according to any one of the aspects of the invention described above, or a tissue or cell derived therefrom.

The screening step may involve measuring the induction of a gene coding for a transcription factor, a drug metabolising enzyme or a drug transporter protein. The screening step may involve measuring the level of expression of a transcription factor, a drug metabolising enzyme or a drug transporter protein or the duration of such expression. The screening step may involve measuring the distribution of expression of a transcription factor, a drug metabolising enzyme or a drug transporter protein.

The assay can be performed in the presence and absence of the drug compound to ascertain differences in distribution, metabolism and toxicity. The effects of the drug compound in the presence and absence of a particular gene or genes can be ascertained by evaluating the effects of the drug compound on different transgenic animals, cells or tissues.

Thus, in a further aspect the invention provides methods for investigating xenobiotic metabolism or toxicity as described herein, comprising administering a drug compound to 2 or more, 3 or more, 4 or more, 5 or more, 6 or more, 7 or more, 8 or more, 9 or more, or 10 or more of the non-human animals, tissues or cells described herein. Preferably, such methods further include a step of comparing the experimental results obtained for different non-human animals, tissues or cells.

More than one drug compound may be administered. For example, a drug compound is considered to activate the CAR transcription factor if the compound mediates induction of the CAR gene. A CAR receptor inverse agonist such as clotrimazole can also administered to an animal expressing the human CAR receptor as a control.

Assays according to further aspects of the invention may provide a screening method for determining whether the metabolism of a first drug compound is modulated by a second drug compound. This method involves administering the first compound in the presence and absence of the second compound to a transgenic animal according to any one of the above-described aspects of the invention, or a tissue or cell derived therefrom, and monitoring for a phenotypic effect. Alternatively, as above, the screening step may involve measuring the induction of a gene, the level, duration or distribution of expression, of a transcription factor, a drug metabolising enzyme or a drug transporter protein. The second compound is determined to modulate the metabolism of the first compound if the second compound effects a change in any one of these tested factors. For example, a physiological effect may be assayed by measuring the toxicity or activity mediated by the administration of the first compound or measuring the half-life of the first drug compound.

In this manner, assays may be used to facilitate the identification of analogs of a drug compound that have reduced or undetectable ability to activate or induce expression of a particular protein, and thus are expected to have fewer side-effects or a longer half-life in vivo.

Various aspects and embodiments of the present invention will now be described in more detail by way of example. It will be appreciated that modification of detail may be made without departing from the scope of the invention.

### BRIEF DESCRIPTION OF THE FIGURES

FIG. 1 depicts the role of nuclear receptors in metabolic pathways within the cell.

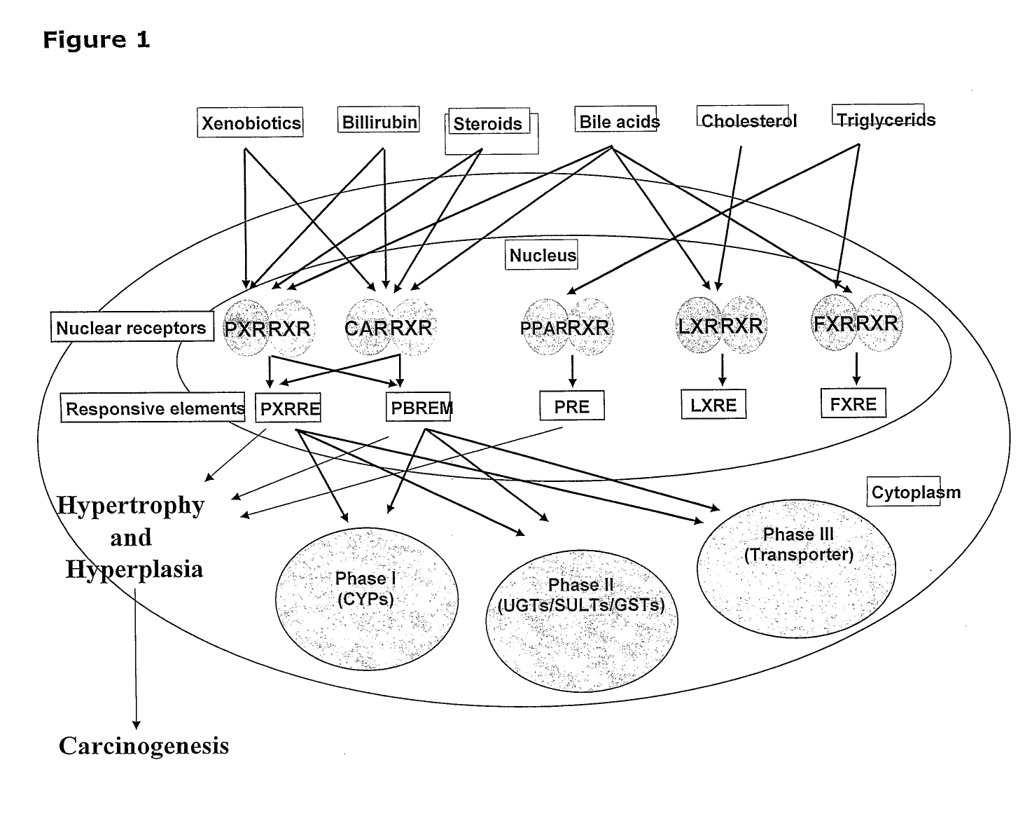

FIG. 2 shows the role of PXR/CAR in the metabolism of non-genotoxic carcinogens.

FIG. 3 shows PXR and CAR mRNA levels in huPXR/huCAR double-humanised mice and in wild-type, huPXR and huCAR mice. Human PXR and CAR mRNA expression is maintained in double humanised mice.

FIG. 4 shows effects of rifampicin and phenobarbital treatment in double-humanised huPXR/huCAR, wild-type, huPXR and huCAR mice, and basal levels of Cyp2b10 and Cyp3a11 protein in huPXR/huCAR, wild-type, huPXR and huCAR mice.

FIG. 5 shows a possible targeting strategy for the PPARα humanisation and knock-out in mice (to produce mice of genotype huPPARα and koPPARα).

FIG. 6 shows a possible targeting strategy for AhR humanisation and knock-out in mice (to produce mice of genotype huAhR and koAhR).

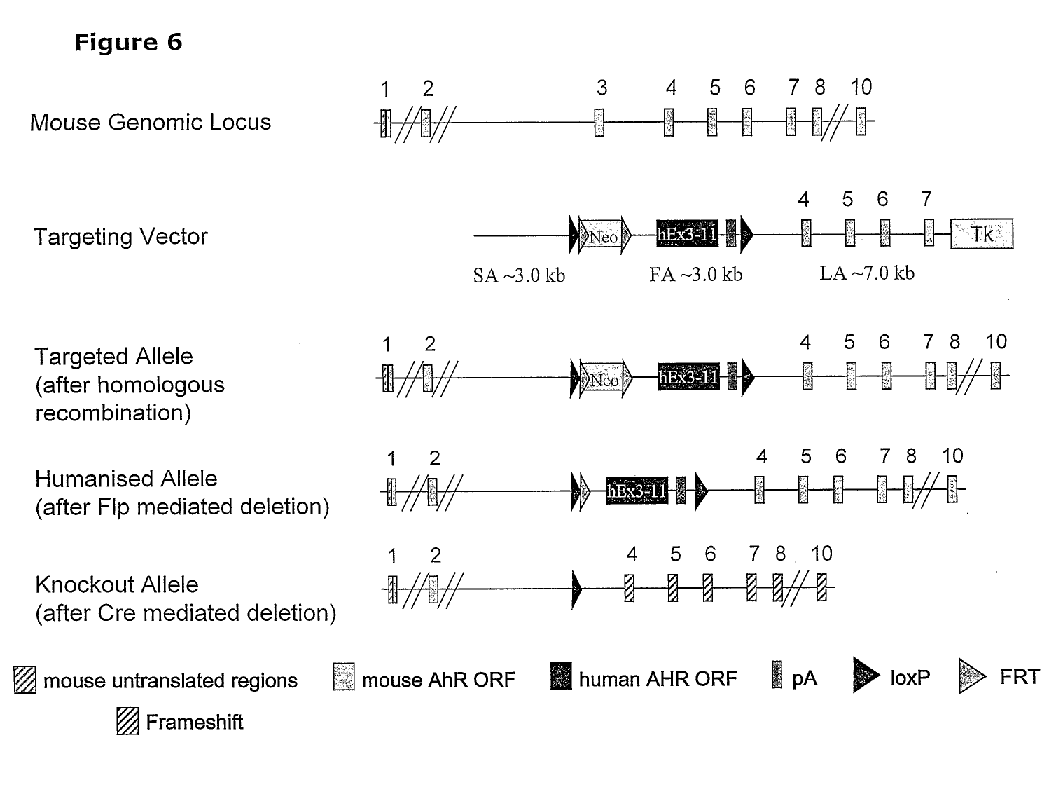

FIG. 7 shows the results of an experiment in which humanised, knockout and wild type mice are exposed to PB treatment. Physiological effects that are monitored include hepatomegaly, P450 induction and hepatocellular proliferation.

FIG. 8 shows the liver/body weight ratios in WT, huPXR/huCAR and PXRKO/CARKO mice. The values are expressed as Mean±SD (% mean own control±% SD; n=3. A Student's t-test (2-sided) was performed on the results; * and ** indicate that the difference is statistically significant from control mice at p<0.05 and p<0.01, respectively.

FIG. 9 shows hepatic S-phase Labelling Indices in PB-treated mice. Osmotic pumps containing BrdU (15 mg/ml/PBS) were implanted into WT, huPXR/huCAR and PXRKO/CARKO mice prior to PB treatment (80 mg/kg/4 days/IP). Livers sections were labelled using a Brdu antibody (Dako). All microscopic images were captured at a magnification of 40×. Data represents random sampling of 10 images per lobes (2) counting approximately 180,000 cells/animal group, as according to Pat-0013-0014. Values are expressed as Mean±SD, n=9-10 for control mice, n=8-9 for PB-treated mice. A Student's t-test (2-sided) was performed on the results with *** indicating that the difference is statistically significant from control mice at p<0.01.

FIG. 10 shows Hepatic apotopic indices in PB-treated mice. Livers sections were labelled using a TUNEL in situ cell detection kit (Roche). All microscopic images were captured at a magnification of 40×. Data represents random sampling of 20 images per lobes (2) counting approximately 380,000 cells/animal group. Values are expressed as Mean±SD, n=9-10 for control mice, n=8-9 for PB-treated mice. A Student's t-test (2-sided) was performed on the results with no statistical significance found.

FIG. 11 shows H&E staining on liver sections taken from control and PB-treated WT, huPXR/huCAR and PXRKO/CARKO mice. Portal vein (P) and central vein (C) are labelled. A 20× objective lens was used to capture the images.

FIG. 12 shows Enzyme activity measurements a) MROD, b) EROD, c) BQ, d) PROD and e) BROD assays. Each assay was performed on individual liver microsomes. Values are expressed as Mean±SD (n=9/10). A Student's t-test (2-sided) was performed on the results; with *, ** and *** statistically different from own control mice at p<0.05, p<0.01 and pO.OOl, respectively.

FIG. 13 shows the effect of PB on hepatic Cyp2b10 and Cyp3a11 protein expression. Liver microsomes (0.3 ug) from each animal were pooled (n=9/10) for wild type, huPXR/huCAR and PXRKO/CARKO mice and characterised for Cyp2b10 and Cyp3a11 by immunoblotting using rabbit polyclonal CH4 (1:2000 dilution) and CH32 antibodies (1:2000 dilution), respectively (C. Henderson, University of Dundee, UK); +ve, control was either purified recombinant his-tagged Cyp3a11 membranes (0.1 pmol/u.l) or purified recombinant his-tagged Cyp2b10 membranes (O.Olpmol/u.l). Blots were developed using ECL and exposed for 30 secs.

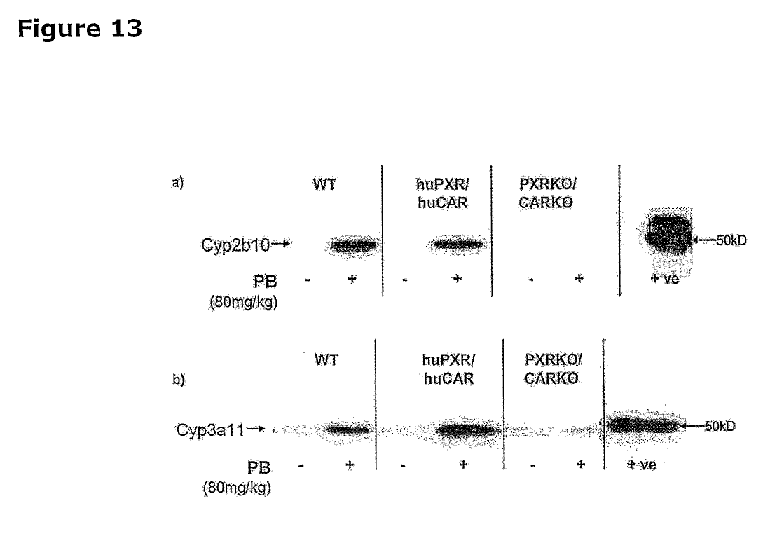

FIG. 14 shows the PXR/CAR dependant species differences in hyperplastic response to Phenobarbital

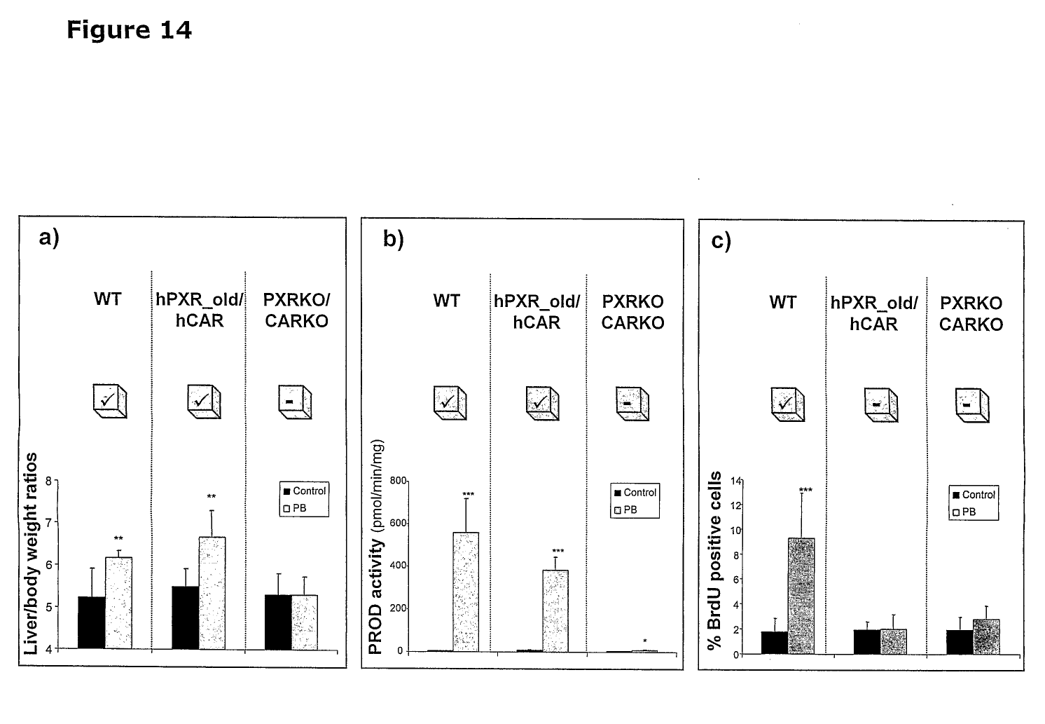

FIG. 15 shows the PXR/CAR dependant species differences in hyperplastic response to Chlordane.

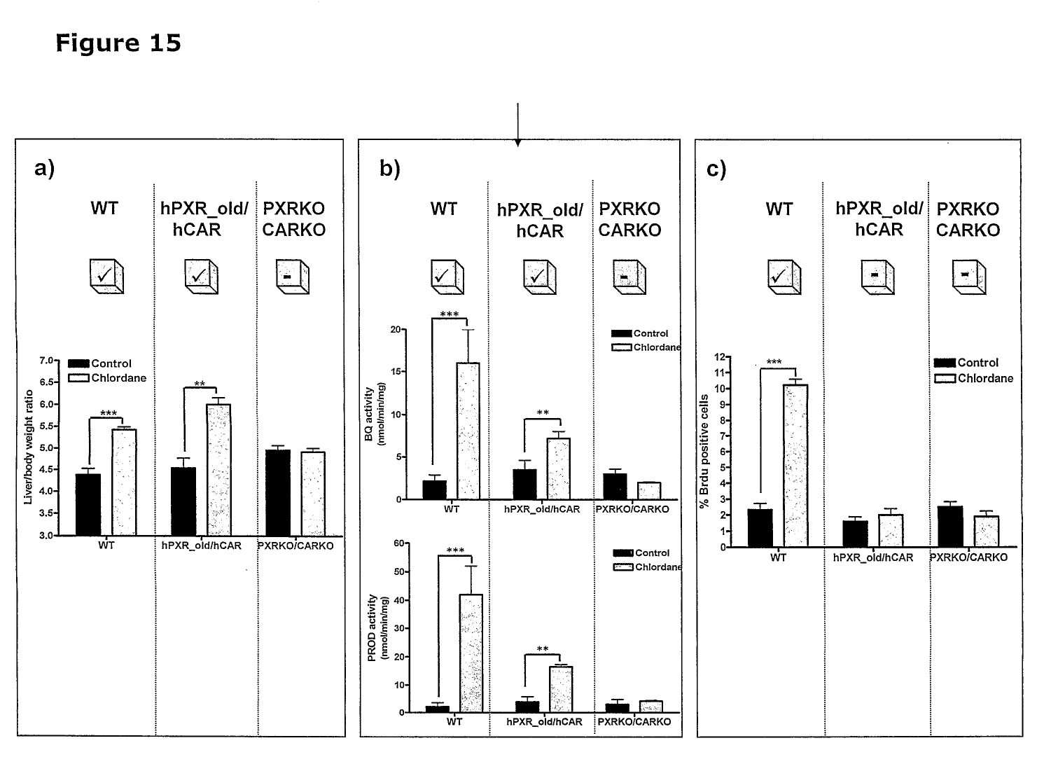

FIG. 16 shows H&E staining on liver sections taken from control and Chlordane-treated WT, hPXR_old/hCAR and PXRKO/CARKO mice. A 20× objective lens was used to capture the images. This figure shows hypertrophy in WT and hPXR_old/hCAR mice but not PXRKO/CARKO upon exposure to Chlordane.

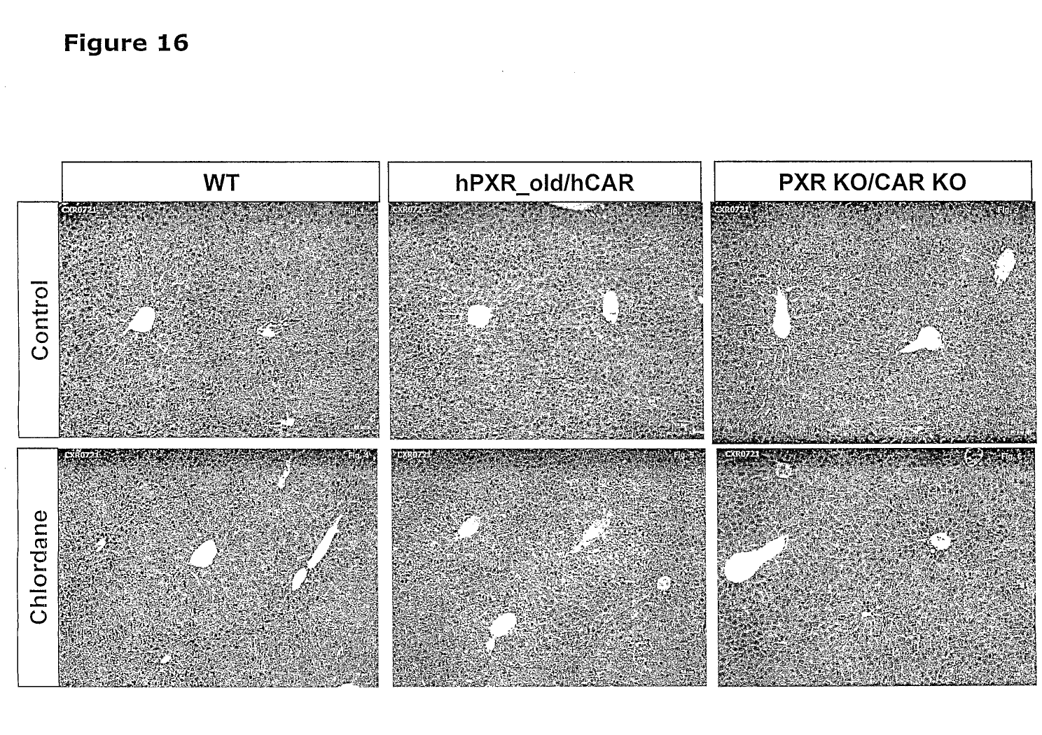

FIG. 17 shows the detection of murine Cyp4a protein by immunoblotting. a) Individual liver microsomes (1 μg protein loaded) from vehicle- and Wy-14,643-treated WT and hPPARα mice were characterised for Cyp4a protein by immunoblotting using a polyclonal goat anti-CYP4A antibody (Dr. C. Henderson, University of Dundee, UK). The standard used was APFO-induced WT rat liver microsomes (1 μg protein loaded).

FIG. 18 shows cyanide-insensitive pCoA oxidation in WT and hPPARα mice. Measurements of cyanide-insensitive pCoA oxidation in crude mitochondrial fractions were performed as described. Values represent reduced NAD+ (pmol/min/mg) from individual mice. hPPARa=hPPARαWy=Wy-14,643-treated, Con=vehicle-treated.

FIG. 19 shows lauric acid hydroxylation in WT and hPPARα mice. Lauric acid hydroxylation activity measurements in liver microsomes were performed. Values represent either formation of (a) 12-OH lauric acid or (b) 11-OH lauric acid (nmol/min/mg) values for individual mice. hPPARα=hPPARαWy=Wy-14,643-treated, Con=vehicle-treated.

FIG. 20 shows hPPARα mRNA structure and locations of primers. M, mouse Exon; H, human Exon; pA, polyA motif.

FIG. 21 shows the presence of human PPARα in the hPPARα mouse by RT-PCR. Liver RNA was isolated from a vehicle-treated WT mouse and a vehicle-treated hPPARα mouse and analysed by RT-PCR using primer pair PPARα-F and PPARα-R. RT-PCR products from the WT mouse were loaded into a single well (lane 1), whereas the RT-PCR products from the hPPARα mouse were loaded in duplicate (lane 2-3). Two bands were detected at 1.4 kb and 1.2 kb in the hPPARα mouse. M=molecular weight marker. N.B. the lower bands in each samples are non-specific.

FIG. 22 shows wild type, splice variant 1 (SV1) and splice variant 2 (SV2) transcripts of human PPARα detected in the hPPARα mouse. SV1 is a transcript with deletion of exon 6, resulting in a frame shift introducing a premature stop codon. SV2 is a transcript using an alternative intronic 3′ splice site resulting in the addition of a 4 bp (GTAG) out-of-frame insertion at the 3′end of exon 5, also resulting in a premature stop codon.

FIG. 23 shows reported full length, splice variant 1 (SV1) and splice variant 2 (SV2) of human PPARα proteins. The alternatively spliced variants SV1 and SV2 encode truncated PPARα proteins containing 174 and 180 amino acids, respectively. The truncation occurs within the ligand binding domain (LBD). The DNA binding domain (DBD) in both truncated protein remains unaltered.

FIG. 24 shows PCR confirmation of triple homozygous PXR/CAR/AhR humanised mice. Mice with the IDs f 241998, f 242001 and f 242004 are triple homozygous humanised for PXR, CAR and AHR.

FIG. 25 shows PCR confirmation of double homozygous CAR/PPARα and heterozygous PXR humanised mice. Mice with the IDs m 245218, f 245221, f 245226 and f 245227 are homozygous humanised for CAR and heterozygous humanised for PXR.

FIG. 26 shows that CAR snRNA is not expressed in the liver or small intestine of CAR knockout mice.

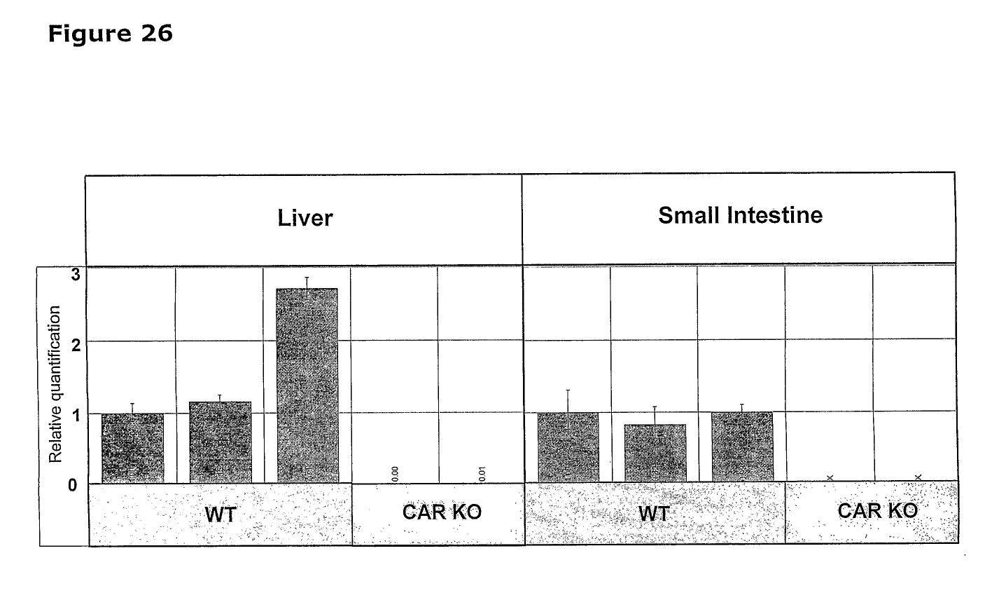

FIG. 27 shows methods of monitoring CAR function, and indicates the species specificity of each method.

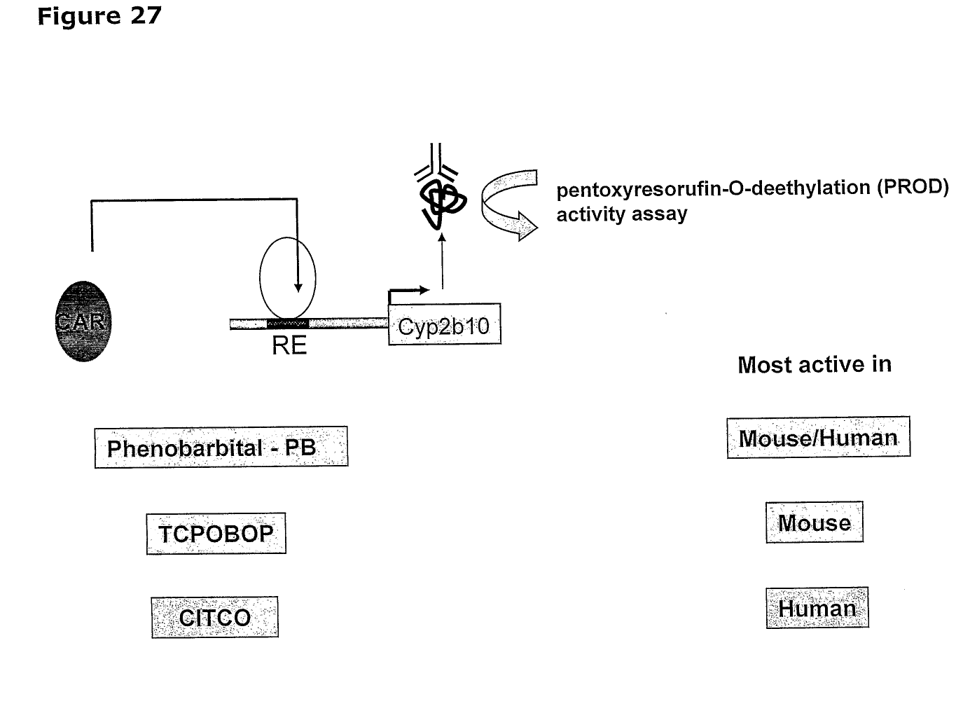

FIG. 28 shows the loss of CAR function in CAR knockout mice, measured using TCPOBOP, which is most active in mice.

FIG. 29 shows the loss of CAR function in CAR knockout mice, measured using CITCO, which is most active in humans.

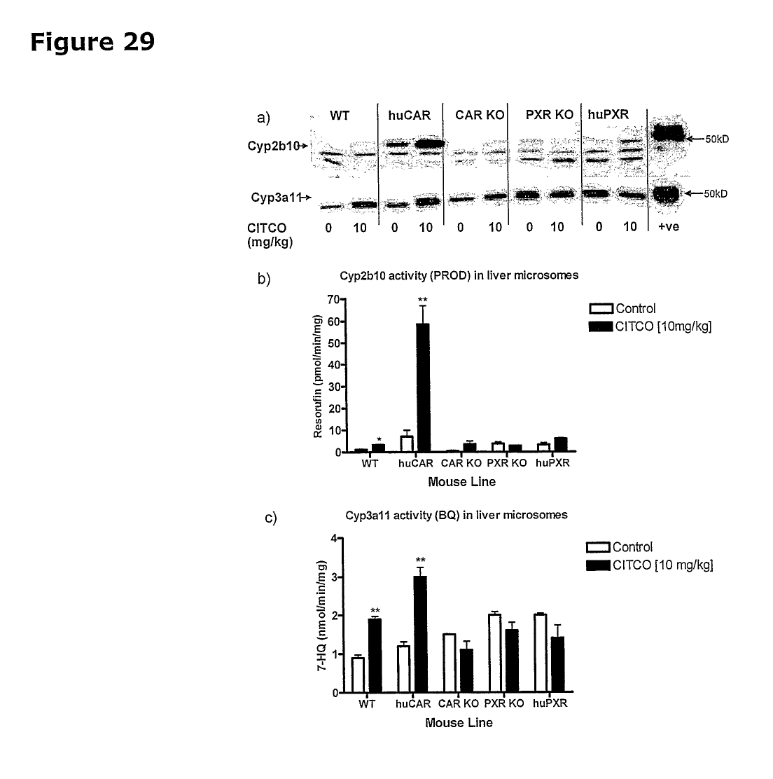

FIG. 30 shows the expression of CAR mRNA in CAR humanised mice. The murine but not the human CAR transcript is expressed in CAR humanised mice. The full length and all human splice variants of CAR are expressed in the CAR humanised mice.

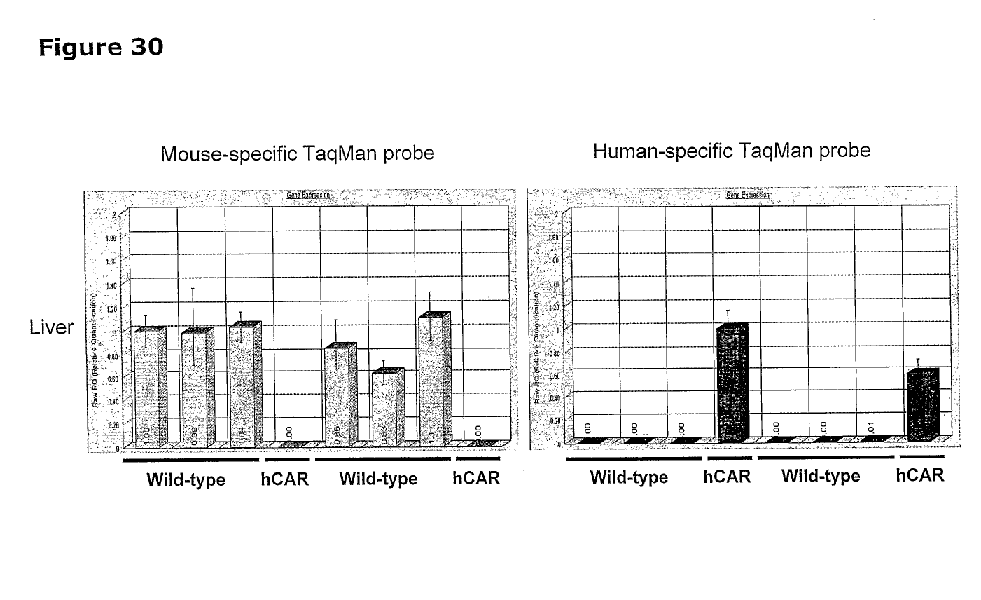

FIG. 31 shows the alternative splicing patterns of human CAR . Two of the ligand binding domain isoforms demonstrate novel functional properties. SV3 has differentially transactivated target gene promoters, and SV2 shows ligand-dependent rather than constitutive interactions with coactivators. Alternative splicing appears to be of the utmost importance for the regulation of CAR expression and function.

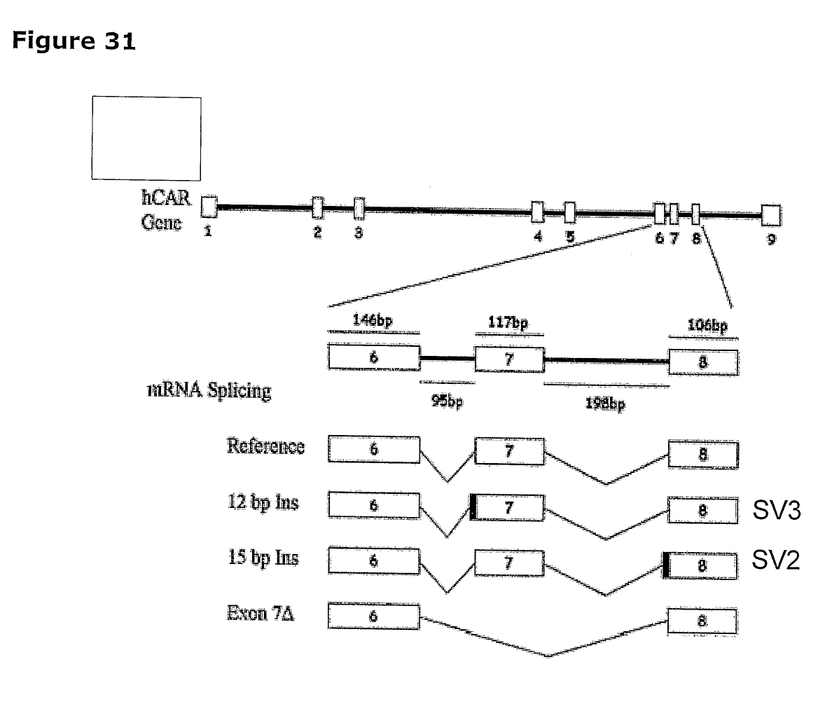

FIG. 32 shows murine CAR dependent P450 induction in wildtype and CAR humanised mice by TCPOBOP, which is most active in mice.

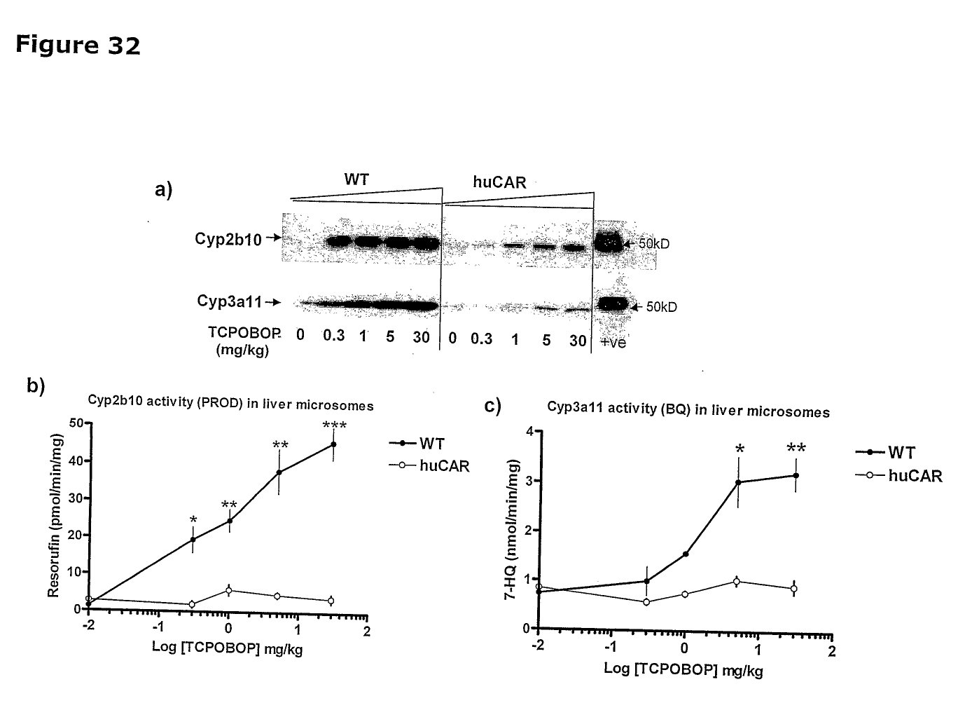

FIG. 33 shows human CAR dependent P450 induction in wildtype and CAR humanised mice by TCPOBOP, which is most active in humans.

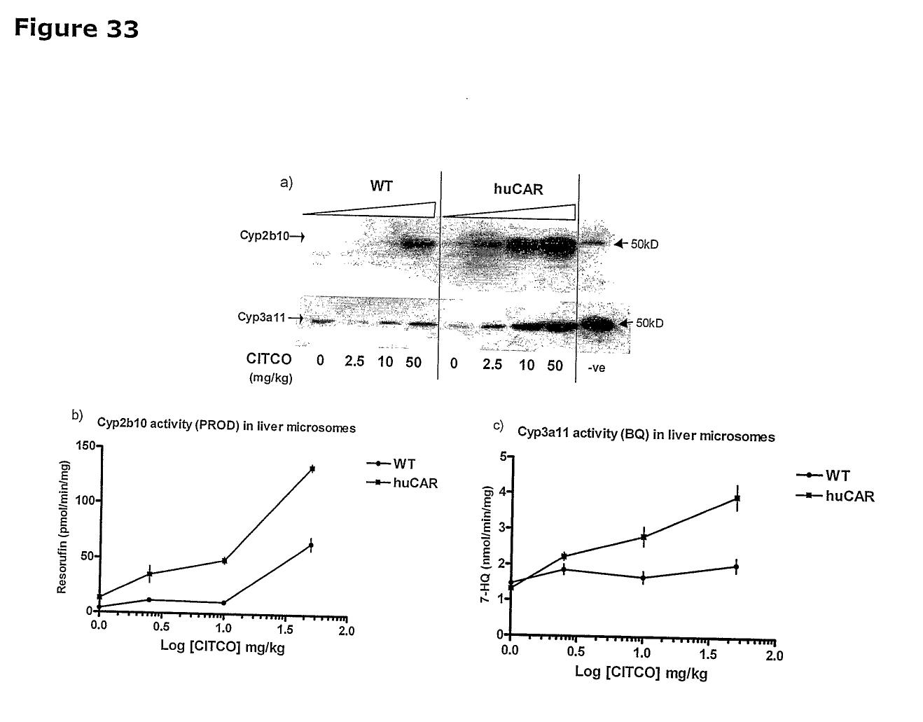

FIG. 34 shows CAR knockout and CAR humanised mice have normal plasma clinical chemistry compared to wildtype mice.

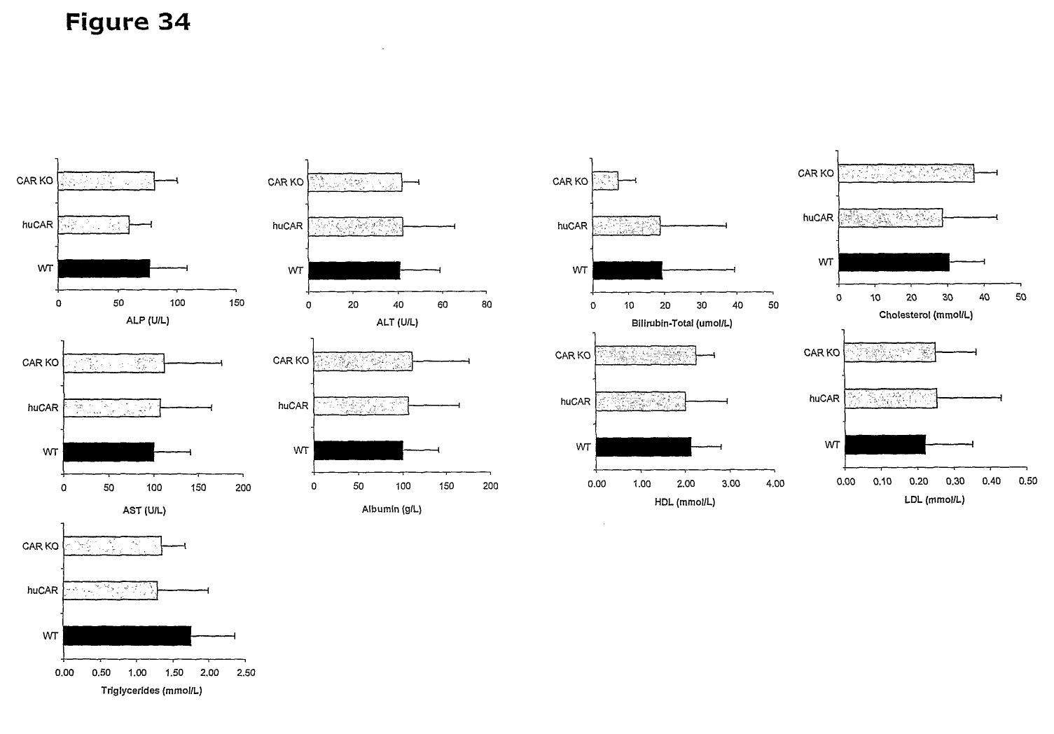

FIG. 35 shows the inductive effect of Phenobarbital in a panel of PXR/CAR knockout and humanised mice. Although Phenobarbital is described as a PXR activator in vitro, the Phenobarbital-mediated activation of Cyp3a11 and Cyp2b10 in vivo is predominantly CAR-dependent.

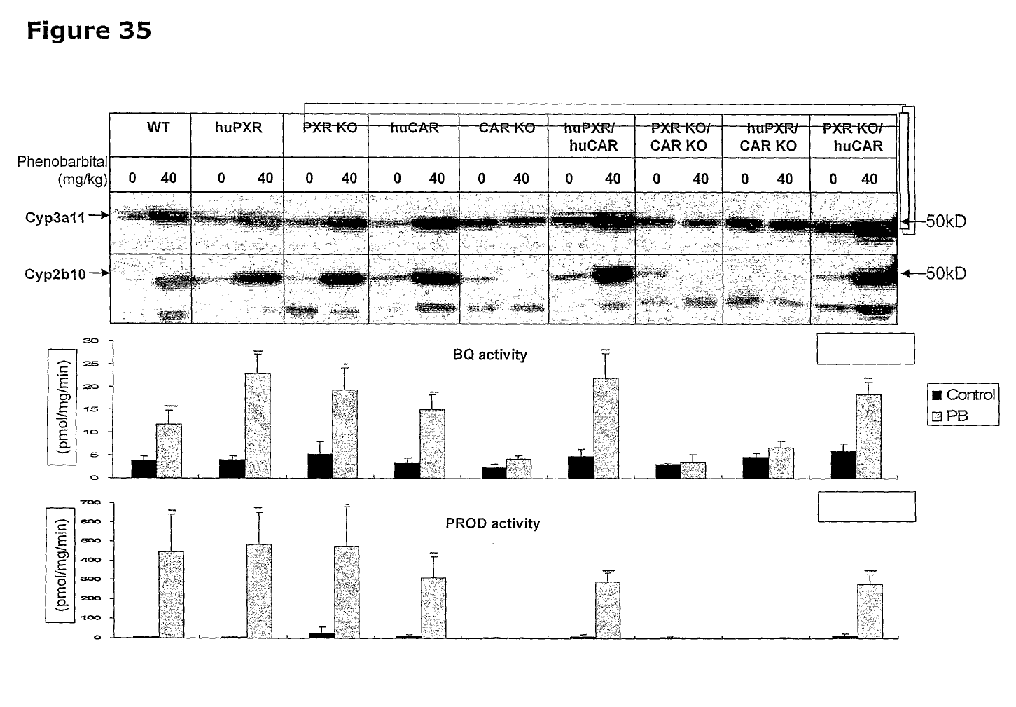

FIG. 36 shows that human CAR supports the hypertrophic, not hyperplastic response to Phenobarbital. A) BrDu hepatocellular labelling; B) H&E analysis on liver sections This highlights the utility of humanised and knockout PXR/CAR mice in assessing the true hazard of non-genotoxic rodent liver growth carcinogens to humans.

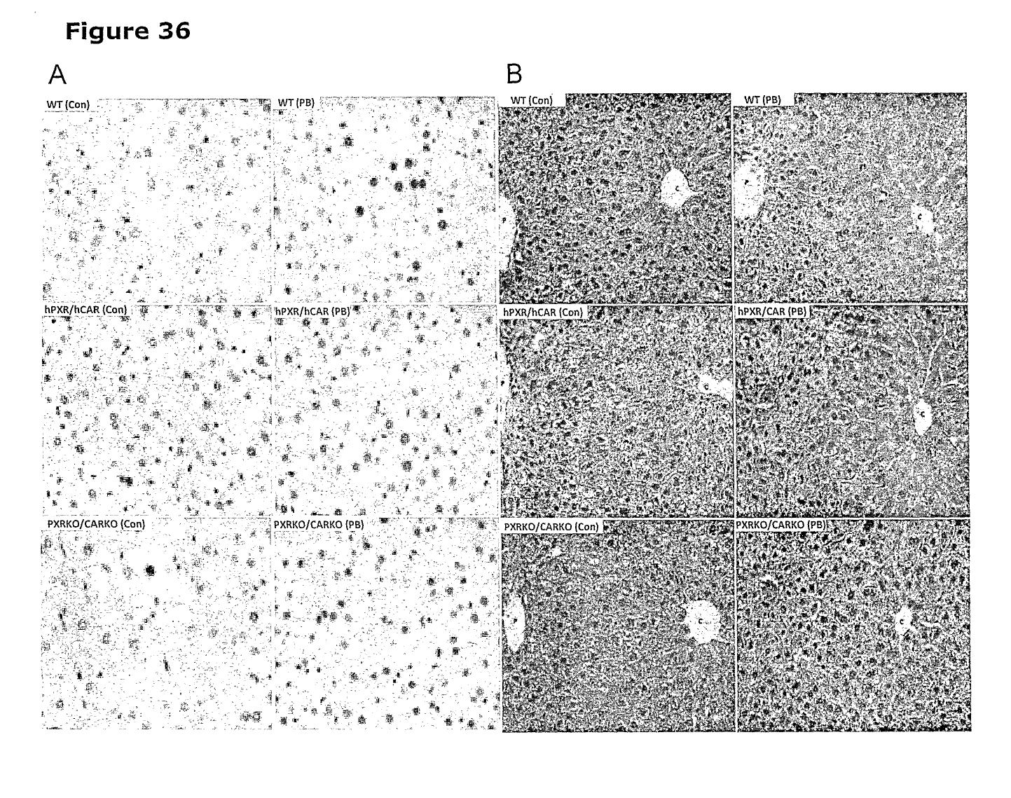

### EXAMPLES

A description of suitable techniques for the generation of mice humanised for both PXR and CAR on a null background can be found in WO2006/064197 (see Examples 3 and 4 and the corresponding Figures).

### Example 1

### PCR Confirmation in Double Homozygous PXR/CAR Humanised Mice that the Murine PXR Gene has Been Exchanged for the Human Countepart

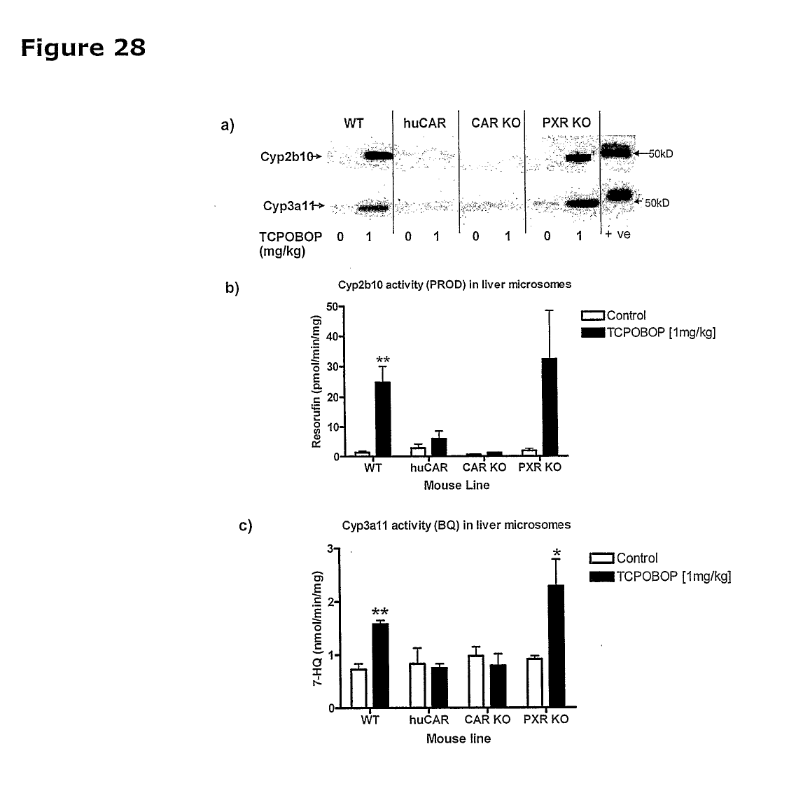

Humanised mice for PXR and CAR (“huPXR/huCAR”) were generated using mice which contained humanised PXR and crossing these into mice which contained humanised CAR to produce mice containing both humanised PXR and humanised CAR. The mice are phenotypically normal following visual inspection. They have been typed using PCR (see FIG. 28 of WO2006/064197) and are homozygously humanised for PXR and CAR. Examples include mice designated “42749” and “42752”.

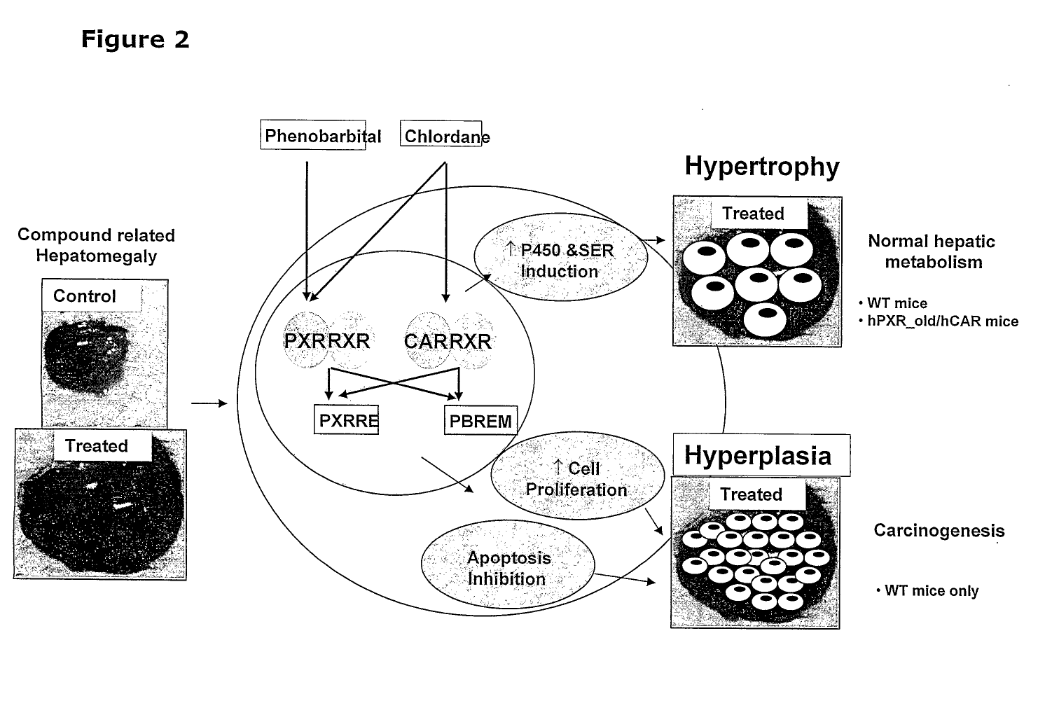

Transcription of PXR and CAR mRNA was quantified by RT-PCR in huPXR/huCAR mice and compared to the relative levels of corresponding mRNA expression in wild-type, huPXR and huCAR mice (FIG. 2). It was thereby confirmed that the huPXR/huCAR mice maintain the levels of human PXR and human CAR expression observed in mice humanised with respect to single genes.

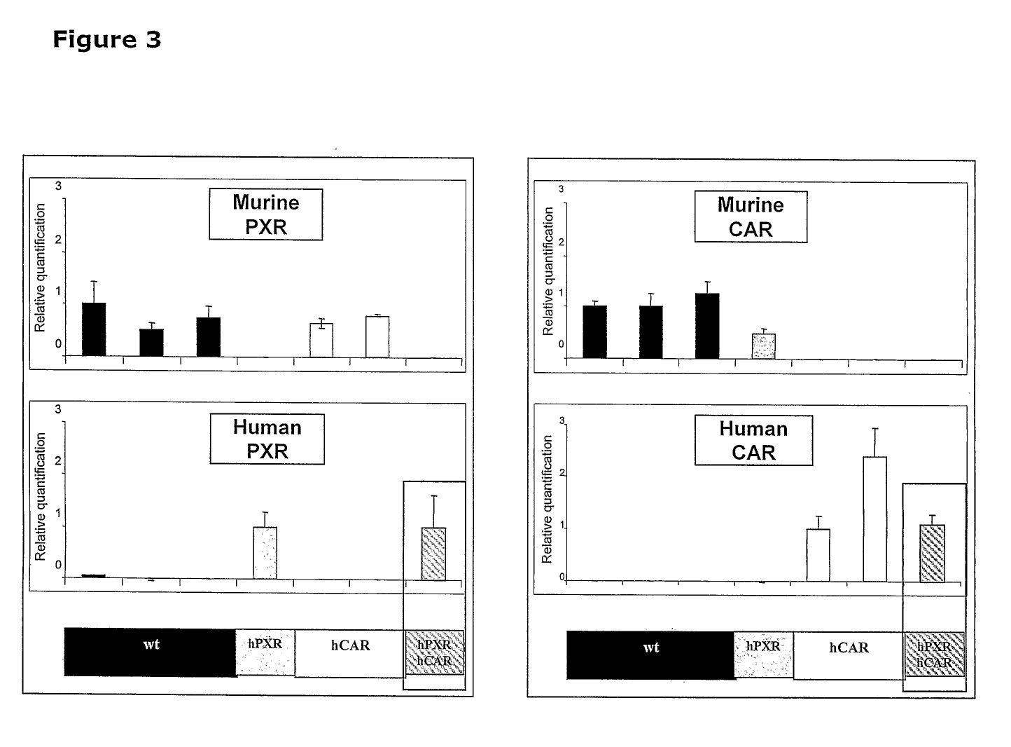

Double-humanised huPXR/huCAR mice, as well as wild-type, huPXR and huCAR mice were treated with the inducers rifampicin and/or phenobarbital. Expression of Cyp2b10 and Cyp3a11 in these inducer-treated mice, as well as in corresponding untreated mice, was visualised and compared by SDS-PAGE followed by Western blotting (FIG. 3). The basal levels of Cyp2b10 and Cyp3a11 in huPXR, huCAR and huPXR/huCAR mice are compared to the basal levels observed in wild-type mice in FIG. 3. This relative quantification shows that basal Cyp2b10 levels increase in the order huPXR→huCAR→huPXR/huCAR. However, basal Cyp3a11 were less markedly increased in huCAR mice. Cyp3a11 levels were increased to an approximately equal extent (more than 2 fold) in both huPXR and double-humanised huPXR/huCAR mice.

Treatment with the human-specific inducer rifampicin led to an increase in the levels of Cyp3a11 in all mice. Whereas the administration of rifampicin and phenobarbital in combination appeared to have no additional effect in the wild type, induction of Cyp3a11 was somewhat stronger in huPXR.

### Example 2

### Pentobarbitone Sleeping Test in Double Homozygous PXR/CAR Humanised Mice

In this experiment, the activity of these transcription factors in combination was determined by measuring the barbiturate induced sleeping time. Sleeping time has been known for many years to be directly proportional to the hepatic cytochrome P450 activity and this activity can be at least in part ascribed to the P450 levels in the liver determined by CAR and PXR function.

Mice were given a single intraperitoneal dose of Narcoren (sodium pentobarbitone; purchased via a Veterinary Consultant; distributed by Merial GmbH, Germany) at 25 mg/kg of body weight. The time taken for the mice to lose, and subsequently to regain, their righting reflex was measured. Results are given in Table 1 below:

TABLE 1
Results of pentobarbitone sleeping test

| | | | | | Sleeping |
|---|---|---|---|---|---|
| Genotype | Sex | Age (weeks) | ID | Weight (g) | time (min) |
| Wt | male | 10 | 42912 | 21.2 | 21 |
| PXR/CAR hum | male | 10 | 42749 | 24.8 | 34 |

Whereas wild type mice given a narcotic dose of pentobarbitone slept for 21 minutes, the double humanised mice for CAR and PXR slept for 34 minutes. These mice therefore demonstrate a significant difference to their wild type controls indicating that the double humanised mouse has a marked difference in its response to drugs relative to the wild type animals.

### Summary of Work in Examples 1 and 2 Above

A model has been developed where human PXR is expressed in both the liver and GI tract of mice in the predicted fashion at levels equivalent to those of the endogenous gene. The PXR protein has been shown to be functional as the mice are responsive to compounds known to induce gene expression via this pathway.

Equivalent humanisation has also been achieved with respect to the CAR gene (huCAR mice). Strain differences between wild type and the humanised mice have been demonstrated and the humanised mice have been shown to be more responsive to compounds known to be more active in humans than in mice, i.e., to human PXR or human CAR rather than murine PXR or murine CAR. The construction of knock-out lines has also been confirmed for both the PXR and the CAR genes (koPXR and koCAR).

Moreover, mice which contained humanised PXR have also been crossed into mice which contained humanised CAR to produce mice containing both humanised PXR and humanised CAR.

### Example 3

### huPPARα and koPPARα

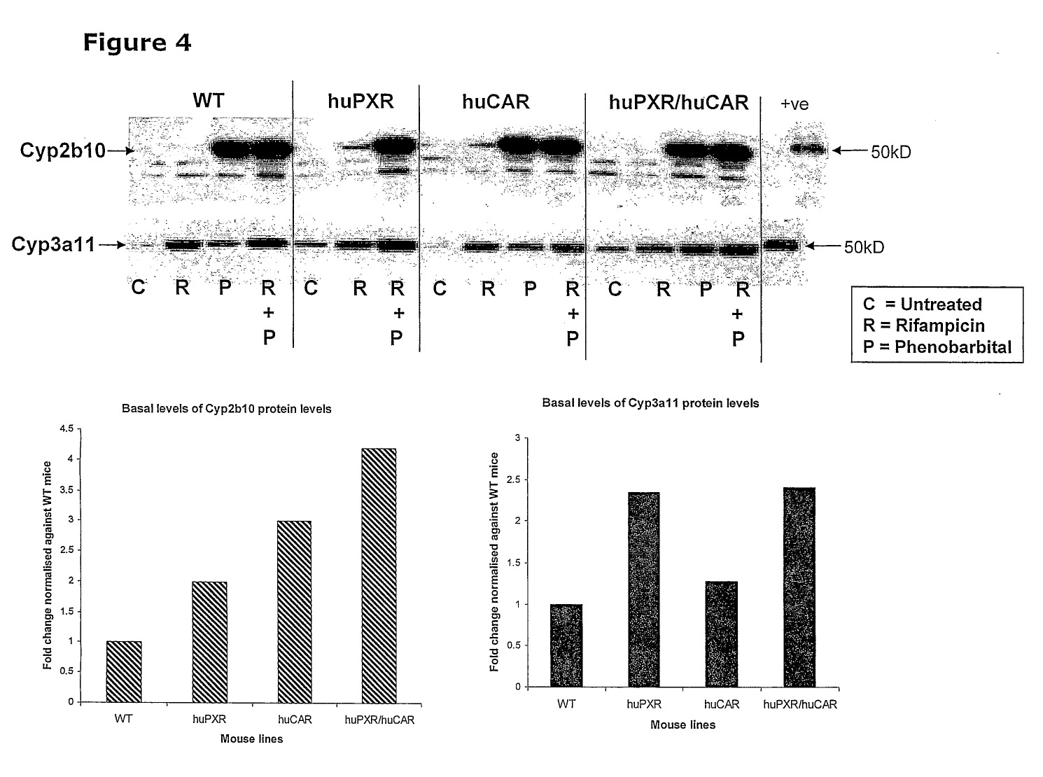

A DNA sequence encoding human PPARα has been inserted into the mouse PPARα locus, as shown in FIG. 4, enabling expression of human PPARα under the control of the mouse PPARα promoter. The DNA sequence encoding human PPARα comprises at least part of intron 5 and intron 6 of the human PPARα gene (FIG. 4). The targeting vector(s) include sequence elements that enable Cre-mediated PPARα knock-out to produce koPPARα (FIG. 4).

### Example 4

### Characterisation of huPPARα and koPPARα Humanised Mice

Six male C57BL/6J mice were obtained from Harlan, (UK). Three male homozygous hPPARα mice were generated according to the protocols herein by TaconicArtemis, Germany. All mice were sexually mature. On arrival in the MSRU the mice were housed on sawdust in solid-bottom, polypropylene cages. No environment enhancing materials was used during treatment.

In the animal room the environment was controlled to provide conditions suitable for the C57BL/6J and transgenic strains of mouse. The temperature was maintained within a range of 19-23° C. and relative humidity within a range of 40-70%. There was a nominal 14-15 air changes per hour. Twelve-hour periods of light were cycled with twelve-hour periods of darkness. For this study no special arrangement of cages was used. The mice were allowed to acclimatise for a minimum of five days following arrival in the test facility.

RM1 pelleted diet (supplied by Special Diet Services Ltd., Stepfield, Witham, Essex, UK) was used. The specification of the diet is held by the MSRU, Dundee. Drinking water was taken from the local supply and provided in bottles. Pelleted diet and drinking water was provided ad libitum prior to and throughout the study.

Body and Liver Weights

Animals were treated either with 4 daily doses of Wy-14,643/corn oil (50 mg/kg, orally) or with corn oil alone, as shown in Table 2. Approximately 24 hr after the last dose, all mice were sacrificed using an increasing concentration of CO2. Liver and plasma were collected for analysis.

TABLE 2
Experimental Design

| | | Mouse | Inducing | Dose | Vehi- | |
|---|---|---|---|---|---|---|
| Grp # | Mouse # | Line | Agent | (mg/kg) | cle | Regimen |
| 1 | 1-3 | WT | — | — | Corn | 4 x orally |
| | | | | | Oil | daily |
| 2 | 4 | hPPARα | — | — | Corn | 4 x orally |
| | | | | | Oil | daily |
| 3 | 5-7 | WT | Wy-14,643 | 50 | Corn | 4 x orally |
| | | | | | Oil | daily |
| 4 | 8-9 | hPPARα | Wy-14,643 | 50 | Corn | 4 x orally |
| | | | | | Oil | daily |

Following treatment with Wy-14,643 or the vehicle (corn oil), the mice were sacrificed and their livers removed and weighed. Body weights, liver weights and liver/body weight ratios of all mice were calculated (Tables 3-5).

Dosing solution was prepared on the day of administration by adding corn oil to the requisite quantity of inducing agent and stirring to obtain a fine suspension. The concentration of inducing agent was of supplied chemical, without any correction for purity. Animals were administered vehicle or inducing agent, orally, as indicated in Table 2. The volume administered was 10 ml/kg bodyweight. This route of administration was chosen for consistency with previously published work.

On the day of termination the mice were weighed, the body weights recorded, and then transferred to a suitable room for post mortem. Approximately 24 hrs after treatment, the mice will be killed by exposure to a rising concentration of CO2.

TABLE 3
Terminal body weights.

| | | | | TERMINAL BODY WEIGHTS | | | | |
|---|---|---|---|---|---|---|---|---|
| Mouse # | Mouse Line | Treatment | Body weights (g) | Mean | SD | % Mean | % SD | T-test |
| 1 | WT | Control | 28 | 27 | 2 | 100 | 7 | |
| 2 | WT | Control | 28 | | | | | |
| 3 | WT | Control | 25 | | | | | |
| 5 | WT | Wy-14,643 | 26 | 26 | 1 | 98 | 4 | 0.45 |
| 6 | WT | Wy-14,643 | 27 | | | | | |
| 7 | WT | Wy-14,643 | 25 | | | | | |
| 4 | hPPARα | Control | 25 | NA | NA | NA | NA | |
| 8 | hPPARα | Wy-14,643 | 24 | NA | NA | NA | NA | NA |
| 9 | hPPARα | Wy-14,643 | 30 | NA | NA | NA | NA | NA |

Values are expressed as individual and/or mean ± SD (% mean own control ± % SD); where n = 3. A Student's t-test (2-sided) was performed on control vs. Wy-14,643-treated WT mice, but no statistically significant difference was found.
NA; not applicable as n < 3.

TABLE 4
Liver weights.

| | | | | LIVER WEIGHTS | | | | | |
|---|---|---|---|---|---|---|---|---|---|
| Mouse # | Mouse Line | Treatment | Liver weights (g) | Mean | SD | % Mean | % SD | T-test | |
| 1 | WT | Control | 1.32 | 1.25 | 0.09 | 100 | 7 | | |
| 2 | WT | Control | 1.29 | | | | | | |
| 3 | WT | Control | 1.15 | | | | | | |
| 5 | WT | Wy-14,643 | 1.68 | 1.69 | 0.02 | 135 | 2 | 0.02 | * |
| 6 | WT | Wy-14,643 | 1.68 | | | | | | |
| 7 | WT | Wy-14,643 | 1.72 | | | | | | |
| 4 | hPPARα | Control | 1.24 | NA | NA | NA | NA | | |
| 8 | hPPARα | Wy-14,643 | 1.43 | NA | NA | NA | NA | NA | |
| 9 | hPPARα | Wy-14,643 | 1.88 | NA | NA | NA | NA | NA | |

Values are expressed as individual and/or mean ± SD (% mean own control ± % SD); where n = 3. A Student's t-test (2-sided) was performed on control vs_{:} Wy-14,643-treated WT mice, revealing a statistically significant difference (* p < 0.05).
NA; not applicable as n < 3.

TABLE 5
Liver:body weight ratios.

| | | | Liver | LIVER BODY | | | | | |
|---|---|---|---|---|---|---|---|---|---|
| | | | body weight | WEIGHT RATIOS (%) | | | | | |
| Mouse # | Mouse Line | Treatment | ratios (%) | Mean | SD | % Mean | % SD | T-test | |
| 1 | WT | Control | 4.73 | 4.66 | 0.08 | 100 | 2 | | |
| 2 | WT | Control | 4.57 | | | | | | |
| 3 | WT | Control | 4.67 | | | | | | |
| 5 | WT | Wy-14,643 | 6.43 | 6.46 | 0.31 | 139 | 7 | 0.01 | ** |
| 6 | WT | Wy-14,643 | 6.16 | | | | | | |
| 7 | WT | Wy-14,643 | 6.78 | | | | | | |
| 4 | hPPARα | Control | 4.90 | NA | NA | NA | NA | | |
| 8 | hPPARα | Wy-14,643 | 5.86 | NA | NA | NA | NA | NA | |
| 9 | hPPARα | Wy-14,643 | 6.34 | NA | NA | NA | NA | NA | |

Values are expressed as individual and/or mean ± SD (% mean own control ± % SD); where n = 3. A Student's t-test (2-sided) was performed on control vs_{:} Wy-14,643-treated WT mice, revealing a statistically significant difference (** p < 0.01).
NA; not applicable as n < 3.

Absolute and relative liver weights were similar between vehicle-treated WT and hPPARα mice and significant increases were detected in both strains following treatment with Wy-14,643. Absolute liver weights were increased following treatment with Wy-14,643 in WT mice by ˜39% and in the two transgenic mice by 20 and 29%, when compared with vehicle-treated mice.

Plasma Clinical Chemistry

The plasma was processed by taking blood from the terminated mice by cardiac puncture into lithium/heparin-coated tubes. Following removal into suitable tubes for plasma preparation, terminal blood samples taken by cardiac puncture were mixed on a roller for 10 min then cooled on ice. Red blood cells were removed by centrifugation (2,000-3,000 rpm for 10 min at 8-10° C.). Immediately after centrifugation, the supernatant (plasma) was collected in eppendorfs and kept on wet ice. Plasma was transferred into cryovials for clinical chemistry and immediately flash frozen in liquid nitrogen, then stored at approximately −70° C.

All clinical chemistry analytes measured in mouse plasma using the COBAS Integra 400+ (Roche) had been optimized for human plasma samples according to manufacturers' instructions. An extensive historical database of clinical chemistry analytes had been generated and was used to verify mouse samples assayed using the COBAS Integra 400+.

In order to investigate potential hepatotoxicity caused by Wy-14,643 treatment, markers of liver damage, alanine aminotransferase (ALT), aspartate aminotransferase (AST) and alkaline phosphatase (ALP) were measured in WT and hPPARα mice (Tables 6-8). Liver related plasma biomarkers, albumin (ALB), conjugated and total bilirubin (BIL-D and BIL-T respectively), cholesterol, high density lipoprotein (HDL-C), low density lipoprotein (LDL-C) and triglycerides were also investigated in all available plasma samples in order to characterise basal hepatic function in hPPARα mice relative to WT mice (Tables 9-15).

Concerning plasma markers of hepatotoxicity, ALT, AST and ALP concentration appeared to be unaltered in both mouse lines following treatment with Wy-14,643; however due to variability within the small treatment groups no clear conclusions could be made concerning Wy-14,643-mediated hepatotoxicity.

TABLE 6
Plasma ALP levels.

| | | | Diluted ALP | Dilution | Normalised | ALP (U/L) | | | | |
|---|---|---|---|---|---|---|---|---|---|---|
| Mouse # | Mouse Line | Treatment | (U/L) | factor | ALP (U/L) | Mean | SD | % Mean | % SD | T-test |
| 1 | WT | Control | 45 | 2 | 90 | 100 | 22 | 100 | 22 | |
| 2 | WT | Control | 42 | 2 | 84 | | | | | |
| 3 | WT | Control | 63 | 2 | 125 | | | | | |
| 5 | WT | Wy-14,643 | 69 | 2 | 138 | 129 | 10 | 129 | 10 | 0.14935 |
| 6 | WT | Wy-14,643 | 59 | 2 | 118 | | | | | |
| 7 | WT | Wy-14,643 | 65 | 2 | 130 | | | | | |
| 4 | hPPARα | Control | 44 | 2 | 88 | NA | NA | NA | NA | |
| 8 | hPPARα | Wy-14,643 | 41 | 2 | 82 | NA | NA | NA | NA | NA |
| 9 | hPPARα | Wy-14,643 | 43 | 2 | 86 | NA | NA | NA | NA | NA |

Values are expressed as individual and/or mean ± SD (% mean own control ± % SD); where n = 3. A Student's t-test (2-sided) was performed on control vs_{:} Wy-14,643-treated WT mice, but no statistically significant difference was found.
NA; not applicable as n < 3.

TABLE 7
Plasma ALT levels.

| | | | Diluted ALT | Dilution | Normalised | ALT (U/L) | | | |
|---|---|---|---|---|---|---|---|---|---|
| Mouse # | Mouse Line | Treatment | (U/L) | factor | ALT (U/L) | Mean | SD | % Mean | % SD |
| 1 | WT | Control | 16 | 2 | 33 | 29 | 9 | 100 | 31 |
| 2 | WT | Control | 18 | 2 | 36 | | | | |
| 3 | WT | Control | 9 | 2 | 19 | | | | |
| 5 | WT | Wy-14,643 | 17 | 2 | 34 | 34 | 2 | 117 | 6 |
| 6 | WT | Wy-14,643 | 16 | 2 | 32 | | | | |
| 7 | WT | Wy-14,643 | 18 | 2 | 36 | | | | |
| 4 | hPPARα | Control | 20 | 2 | 39 | NA | NA | NA | NA |
| 8 | hPPARα | Wy-14,643 | 18 | 2 | 37 | NA | NA | NA | NA |
| 9 | hPPARα | Wy-14,643 | 15 | 2 | 29 | NA | NA | NA | NA |

Values are expressed as individual and/or mean ± SD (% mean own control ± % SD); where n = 3. A Student's t-test (2-sided) was performed on control vs_{:} Wy-14,643-treated WT mice, but no statistically significant difference was found.
NA; not applicable as n < 3.

TABLE 8
Plasma AST levels.

| | | | Diluted | Dilution | Normalised | AST (U/L) | | | | |
|---|---|---|---|---|---|---|---|---|---|---|
| Mouse # | Mouse Line | Treatment | AST (U/L) | factor | AST (U/L) | Mean | SD | % Mean | % SD | T-test |
| 1 | WT | Control | 67 | 2 | 133 | 98 | 35 | 100 | 36 | |
| 2 | WT | Control | 50 | 2 | 100 | | | | | |
| 3 | WT | Control | 31 | 2 | 62 | | | | | |
| 5 | WT | Wy-14,643 | 79 | 2 | 158 | 119 | 76 | 121 | 77 | 0.71266 |
| 6 | WT | Wy-14,643 | 16 | 2 | 32 | | | | | |
| 7 | WT | Wy-14,643 | 84 | 2 | 168 | | | | | |
| 4 | hPPARα | Control | 95 | 2 | 190 | NA | NA | NA | NA | |
| 8 | hPPARα | Wy-14,643 | 141 | 2 | 281 | NA | NA | NA | NA | NA |
| 9 | hPPARα | Wy-14,643 | 27 | 2 | 55 | NA | NA | NA | NA | NA |

Values are expressed as individual and/or mean ± SD (% mean own control ± % SD); where n = 3. A Student's t-test (2-sided) was performed on control vs_{:} Wy-14,643-treated WT mice, but no statistically significant difference was found.
NA; not applicable as n < 3.

Plasma albumin levels were unaltered in both mouse lines following treatment with Wy-14,643. On the limited number of mice available to analyse, detectable bilirubin concentrations were variable between animals and treatment groups. Plasma LDL, HDL and cholesterol levels were unchanged in hPPARα mice when compared with WT mice, irrespective of treatment. However, following treatment with Wy-14,643, triglyceride concentrations were significantly decreased by ˜50% (p<0.05) in WT mice and by 25 and 29% in the two hPPARα mice, compared to the levels seen in the corresponding control mice. Interestingly, triglyceride levels were 80% greater in the single vehicle-treated transgenic mouse compared to those in vehicle-treated WT mice (n=3).

TABLE 9
Plasma albumin levels.

| | | | Diluted | Dilution | Normalised | Albumin (g/L) | | | | |
|---|---|---|---|---|---|---|---|---|---|---|
| Mouse # | Mouse Line | Treatment | ALB (g/L) | factor | ALB (g/L) | Mean | SD | % Mean | % SD | T-test |
| 1 | WT | Control | 18 | 2 | 36 | 37 | 1 | 100 | 4 | |
| 2 | WT | Control | 19 | 2 | 39 | | | | | |
| 3 | WT | Control | 19 | 2 | 38 | | | | | |
| 5 | WT | Wy-14,643 | 20 | 2 | 41 | 38 | 3 | 101 | 8 | 0.87574 |
| 6 | WT | Wy-14,643 | 19 | 2 | 39 | | | | | |
| 7 | WT | Wy-14,643 | 17 | 2 | 34 | | | | | |
| 4 | hPPARα | Control | 19 | 2 | 39 | NA | NA | NA | NA | |
| 8 | hPPARα | Wy-14,643 | 18 | 2 | 36 | NA | NA | NA | NA | NA |
| 9 | hPPARα | Wy-14,643 | 20 | 2 | 40 | NA | NA | NA | NA | NA |

Values are expressed as individual and/or mean ± SD (% mean own control ± % SD); where n = 3. A Student's t-test (2-sided) was performed on control vs_{:} Wy-14,643-treated WT mice, but no statistically significant difference was found.
NA; not applicable as n < 3.

TABLE 10
Plasma conjugated bilirubin (BIL-D) levels.

| | | | Diluted | | Normalised | | | | | |
|---|---|---|---|---|---|---|---|---|---|---|
| | | | BIL-D | Dilution | BIL-D | BIL-D (μmol/L) | | | | |
| Mouse # | Mouse Line | Treatment | (μmol/L) | factor | (μmol/L) | Mean | SD | % Mean | % SD | T-test |
| 1 | WT | Control | ND | 2 | ND | 0.37 | 0.30 | 100 | 80 | |
| 2 | WT | Control | 0.08 | 2 | 0.16 | | | | | |
| 3 | WT | Control | 0.29 | 2 | 0.58 | | | | | |
| 5 | WT | Wy-14,643 | 0.07 | 2 | 0.14 | 0.08 | 0.05 | 22 | 14 | NA |
| 6 | WT | Wy-14,643 | 0.03 | 2 | 0.06 | | | | | |
| 7 | WT | Wy-14,643 | 0.02 | 2 | 0.04 | | | | | |
| 4 | hPPARα | Control | ND | 2 | ND | NA | NA | NA | NA | |
| 8 | hPPARα | Wy-14,643 | 0.05 | 2 | 0.10 | NA | NA | NA | NA | NA |
| 9 | hPPARα | Wy-14,643 | 0.07 | 2 | 0.14 | NA | NA | NA | NA | NA |

Values are expressed as individual and/or mean ± SD (% mean own control ± % SD); where n = 3. A Student's t-test (2-sided) was performed on control vs_{:} Wy-14,643-treated WT mice, but no statistically significant difference was found.
NA; not applicable as n < 3,
ND; not detectable (below limit of assay detection).

TABLE 11
Plasma total bilirubin (BIL-T) levels.

| | | | Diluted | | Normalised | | | | | |
|---|---|---|---|---|---|---|---|---|---|---|
| | | | BIL-T | Dilution | BIL-T | BIL-T (μmol/L) | | | | |
| Mouse # | Mouse Line | Treatment | (μmol/L) | factor | (μmol/L) | Mean | SD | % Mean | % SD | T-test |
| 1 | WT | Control | 12 | 2 | 23 | 24 | 1 | 100 | 6 | |
| 2 | WT | Control | 13 | 2 | 26 | | | | | |
| 3 | WT | Control | 12 | 2 | 24 | | | | | |
| 5 | WT | Wy-14,643 | 11 | 2 | 23 | 17 | 6 | 69 | 24 | 0.1739 |
| 6 | WT | Wy-14,643 | 8 | 2 | 16 | | | | | |
| 7 | WT | Wy-14,643 | 6 | 2 | 11 | | | | | |
| 4 | hPPARα | Control | 19 | 2 | 38 | NA | NA | NA | NA | |
| 8 | hPPARα | Wy-14,643 | 6 | 2 | 12 | NA | NA | NA | NA | NA |
| 9 | hPPARα | Wy-14,643 | 16 | 2 | 32 | NA | NA | NA | NA | NA |

Values are expressed as individual and/or mean SD (% mean own control ± % SD); where n = 3. A Student's t-test (2-sided) was performed on control vs_{:} Wy-14,643-treated WT mice, but no statistically significant difference was found.
NA; not applicable as n < 3.

TABLE 12
Plasma cholesterol levels.

| | | | Diluted | | Normalised | | | | | |
|---|---|---|---|---|---|---|---|---|---|---|
| | | | Chol | Dilution | Chol | Cholesterol (mmol/L) | | | | |
| Mouse # | Mouse Line | Treatment | (mmol/L) | factor | (mmol/L) | Mean | SD | % Mean | % SD | T-test |
| 1 | WT | Control | 1.22 | 2 | 2.44 | 2.53 | 0.09 | 100 | 4 | |
| 2 | WT | Control | 1.31 | 2 | 2.62 | | | | | |
| 3 | WT | Control | 1.26 | 2 | 2.52 | | | | | |
| 5 | WT | Wy-14,643 | 1.25 | 2 | 2.50 | 2.45 | 0.38 | 97 | 15 | 0.7858 |
| 6 | WT | Wy-14,643 | 1.02 | 2 | 2.04 | | | | | |
| 7 | WT | Wy-14,643 | 1.40 | 2 | 2.80 | | | | | |
| 4 | hPPARα | Control | 1.35 | 2 | 2.70 | NA | NA | NA | NA | |
| 8 | hPPARα | Wy-14,643 | 1.18 | 2 | 2.36 | NA | NA | NA | NA | NA |
| 9 | hPPARα | Wy-14,643 | 1.47 | 2 | 2.94 | NA | NA | NA | NA | NA |

Values are expressed as individual and/or mean ± SD (% mean own control ± % SD); where n = 3. A Student's t-test (2-sided) was performed on control vs_{:} Wy-14,643-treated WT mice, but no statistically significant difference was found.
NA; not applicable as n < 3.

TABLE 13
Plasma HDL levels.

| | | | Diluted | | Normalised | | | | | |
|---|---|---|---|---|---|---|---|---|---|---|
| | | | HDL | Dilution | HDL | HDL (mmol/L) | | | | |
| Mouse # | Mouse Line | Treatment | (mmol/L) | factor | (mmol/L) | Mean | SD | % Mean | % SD | T-test |
| 1 | WT | Control | 1.20 | 2 | 2.40 | 2.36 | 0.03 | 100 | 1 | |
| 2 | WT | Control | 1.17 | 2 | 2.34 | | | | | |
| 3 | WT | Control | 1.17 | 2 | 2.34 | | | | | |
| 5 | WT | Wy-14,643 | 1.28 | 2 | 2.56 | 2.44 | 0.36 | 103 | 15 | 0.72825 |
| 6 | WT | Wy-14,643 | 1.02 | 2 | 2.04 | | | | | |
| 7 | WT | Wy-14,643 | 1.36 | 2 | 2.72 | | | | | |
| 4 | hPPARα | Control | 1.18 | 2 | 2.36 | NA | NA | NA | NA | |
| 8 | hPPARα | Wy-14,643 | 1.14 | 2 | 2.28 | NA | NA | NA | NA | NA |
| 9 | hPPARα | Wy-14,643 | 1.46 | 2 | 2.92 | NA | NA | NA | NA | NA |

Values are expressed as individual and/or mean ± SD (% mean own control ± % SD); where n = 3. A Student's t-test (2-sided) was performed on control vs_{:} Wy-14,643-treated WT mice, but no statistically significant difference was found.
NA; not applicable as n < 3.

TABLE 14
Plasma LDL levels.

| | | | Diluted | | Normalised | | | | | |
|---|---|---|---|---|---|---|---|---|---|---|
| | | | LDL | Dilution | LDL | LDL (mmol/L) | | | | |
| Mouse # | Mouse Line | Treatment | (mmol/L) | factor | (mmol/L) | Mean | SD | % Mean | % SD | T-test |
| 1 | WT | Control | 0.08 | 2 | 0.16 | 0.25 | 0.09 | 100 | 36 | |
| 2 | WT | Control | 0.17 | 2 | 0.34 | | | | | |
| 3 | WT | Control | 0.13 | 2 | 0.26 | | | | | |
| 5 | WT | Wy-14,643 | 0.12 | 2 | 0.24 | 0.23 | 0.06 | 89 | 24 | 0.76672 |
| 6 | WT | Wy-14,643 | 0.08 | 2 | 0.16 | | | | | |
| 7 | WT | Wy-14,643 | 0.14 | 2 | 0.28 | | | | | |
| 4 | hPPARα | Control | 0.11 | 2 | 0.22 | NA | NA | NA | NA | |
| 8 | hPPARα | Wy-14,643 | 0.07 | 2 | 0.14 | NA | NA | NA | NA | NA |
| 9 | hPPARα | Wy-14,643 | 0.11 | 2 | 0.22 | NA | NA | NA | NA | NA |

Values are expressed as individual and/or mean ± SD (% mean own control ± % SD); where n = 3. A Student's t-test (2-sided) was performed on control vs_{:} Wy-14,643-treated WT mice, but no statistically significant difference was found.
NA; not applicable as n < 3.

TABLE 15
Plasma triglyceride levels.

| | | | Diluted | | Normalised | | | | | | |
|---|---|---|---|---|---|---|---|---|---|---|---|
| | | | Trig | Dilution | Trig | Triglycerides (mmol/L) | | | | | |
| Mouse # | Mouse Line | Treatment | (mmol/L) | factor | (mmol/L) | Mean | SD | % Mean | % SD | T-test | |
| 1 | WT | Control | 0.42 | 2 | 0.84 | 0.95 | 0.09 | 100 | 10 | | |
| 2 | WT | Control | 0.50 | 2 | 1.00 | | | | | | |
| 3 | WT | Control | 0.50 | 2 | 1.00 | | | | | | |
| 5 | WT | Wy-14,643 | 0.27 | 2 | 0.54 | 0.53 | 0.10 | 56 | 11 | 0.04 | * |
| 6 | WT | Wy-14,643 | 0.21 | 2 | 0.42 | | | | | | |
| 7 | WT | Wy-14,643 | 0.31 | 2 | 0.62 | | | | | | |
| 4 | hPPARα | Control | 0.85 | 2 | 1.70 | NA | NA | NA | NA | | |
| 8 | hPPARα | Wy-14,643 | 0.64 | 2 | 1.28 | NA | NA | NA | NA | NA | |
| 9 | hPPARα | Wy-14,643 | 0.60 | 2 | 1.20 | NA | NA | NA | NA | NA | |

Values are expressed as individual and/or mean ± SD (% mean own control ± % SD); where n = 3. A Student's t-test (2-sided) was performed on control vs_{:} Wy-14,643-treated WT mice, which revealed a statistically significant difference (* p < 0.05).
NA; not applicable as n < 3.

Liver Removal and Processing

The liver was processed as follows. The gall bladder was removed, and then the t liver was removed and weighed. Three pieces of liver (5 mm³) were removed and placed in separate cryovials, then flash frozen in liquid nitrogen at approximately −70° C. for Taqman® analysis and DNA sequencing.

Two samples of liver, approximately 2 mm strips, were taken, one from the Left lobe and one from the Median lobe. These were placed in the same scintillation vial containing 20 ml of 10% neutral buffered formalin (NBF) for histology analysis.

The liver was weighed again, and then placed into ice cold 1.15% (w/v) KCl prior to homogenisation and subcellular fractionation.

Fresh weighed livers were processed, according to a modified version of CXR Laboratory Method Sheet (LMS) Cent-001, to homogenate, nuclear, mitochondrial (heavy pellet), cytosolic and microsomal fractions. The method was modified to allow for the collection of nuclear fractions. Fresh liver samples were processed to 10% (v/v) homogenates as according to Cent-001. Homogenates were placed 15 ml Falcon tubes and centrifuged at 50 g for 5 mins at 4° C. to remove cell debris. The remaining supernatant was transferred into 10 ml centrifuge tubes and the pellet was discarded. The supernatant was topped up to 10 ml with ice cold SET buffer then centrifuged at 700 g for 10 mins at 4° C. The resulting supernatant was retained for further fractionation to mitochondrial, cytosolic and microsomal fractions.

The pellet (unwashed crude nuclear membranes) was resuspended in 10 ml ice-cold SET buffer, homogenised with 2-3 passes and then centrifuged at 700 g for 10 mins at 4° C. The resultant supernatant was discarded and the pellet (washed crude nuclear membranes) was resuspended in 1 mL/g original tissue ice-cold SET buffer and homogenised by 2 passes. All fractions were stored at approximately −70° C. prior to analysis.

Following fixation, both liver sections from each animal were processed then paraffin embedded. Wax blocks will be stored at room temperature

Immunoblot Analysis of Cyp4a Protein Expression

To investigate the downstream effects of PPARα activation, the expression of Cyp4a proteins was evaluated by immunoblot analysis in liver microsomes from WT and hPPARα mice.

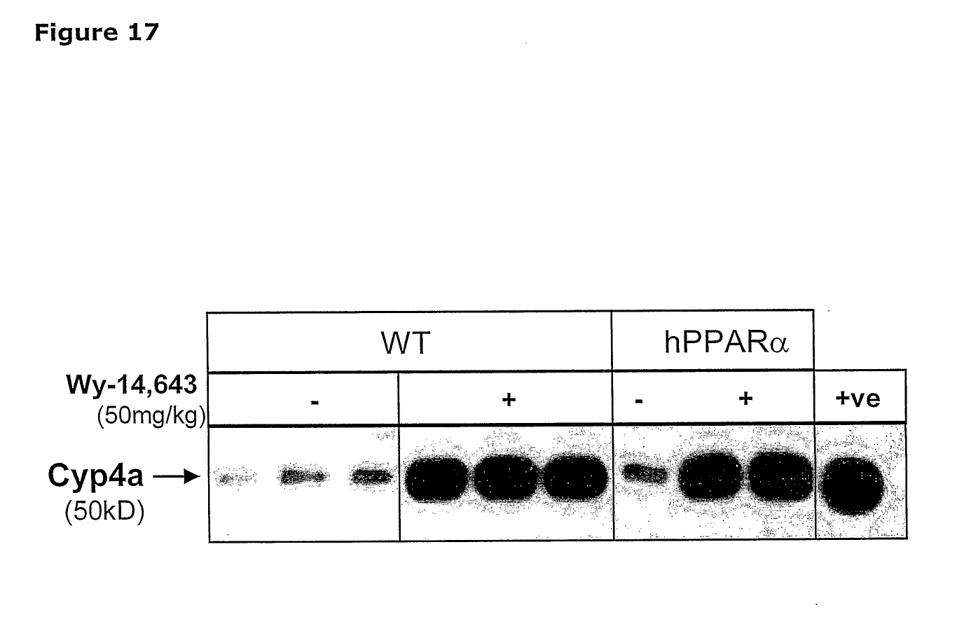

Individual liver microsomal samples were analysed by immunoblotting for Cyp4a. Protein concentration was measured using the CXR microlowry method. Quantification of Cyp4a protein in mouse liver microsomes was carried out. A murine his-tagged Cyp4a recombinant standard was loaded onto the NU-PAGE gel. The results are shown in FIG. 17. The presence of a signal at approximately 50 kD in a positive control sample (ammonium perfluorooctanoate (APFO)-induced rat liver microsomes) demonstrated that the antibody used recognised a protein of the correct molecular weight.

An immunoreactive band specific to Cyp4a was detectable at 50 kD in both vehicle-treated WT and hPPARα mice. Following treatment with Wy-14,643, marked induction was observed in WT mice, consistent with upregulation of Cyp4a protein expression. A similar effect was seen in the hPPARα mice. This is consistent with the hypothesis that human and murine PPARαs have similar affinities for Wy-14,643.

Determination of Cyanide-Insensitive Palmitoyl CoA Oxidation in Mouse Liver

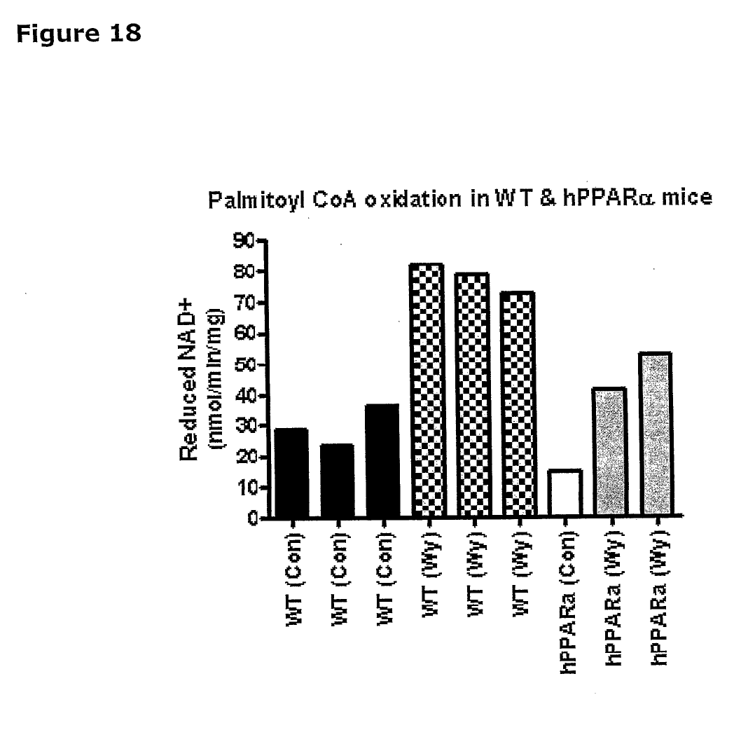

The effect of Wy-14,643 administration on cyanide-insensitive palmitoyl-CoA oxidation was investigated for the heavy pellet fraction (crude mitochondria) from each available mouse liver as a marker of peroxisomal enzyme activity, known to be regulated by PPARα. This assay was used to confirm the presence of a functional PPARα gene in WT and hPPARα mice. Wy-14,643 induced cyanide-insensitive pCoA oxidation in crude mitochondrial fractions from both mouse lines, exhibiting approximately 3-fold induction relative to vehicle controls (FIG. 18, Table 16).

TABLE 15
PCO oxidation in WT and hPPARα mice.

| | | | NAD + red. | | | | | | |
|---|---|---|---|---|---|---|---|---|---|
| Mouse # | Mouse line | Treatment | (nmol/min/mg) | MEAN | SD | % MEAN | % SD | T-test | |
| 1 | WT | Control | 29 | 29 | 6 | 100 | 22 | | |
| 2 | WT | Control | 24 | | | | | | |
| 3 | WT | Control | 36 | | | | | | |
| 5 | WT | Wy-14,643 | 82 | 78 | 5 | 265 | 16 | 0.01 | ** |
| 6 | WT | Wy-14,643 | 79 | | | | | | |
| 7 | WT | Wy-14,643 | 73 | | | | | | |
| 4 | hPPARα | Control | 15 | NA | NA | NA | NA | | |
| 8 | hPPARα | Wy-14,643 | 41 | NA | NA | NA | NA | NA | |
| 9 | hPPARα | Wy-14,643 | 53 | NA | NA | NA | NA | NA | |

Values are expressed as individual and/or mean ± SD (% mean own control ± % SD); where n = 3. A Student's t-test (2-sided) was performed on control vs_{:} Wy-14,643-treated WT mice, which revealed a statistically significant difference (** p < 0.01).
NA; not applicable as n < 3.

Lauric Acid Hydroxylation

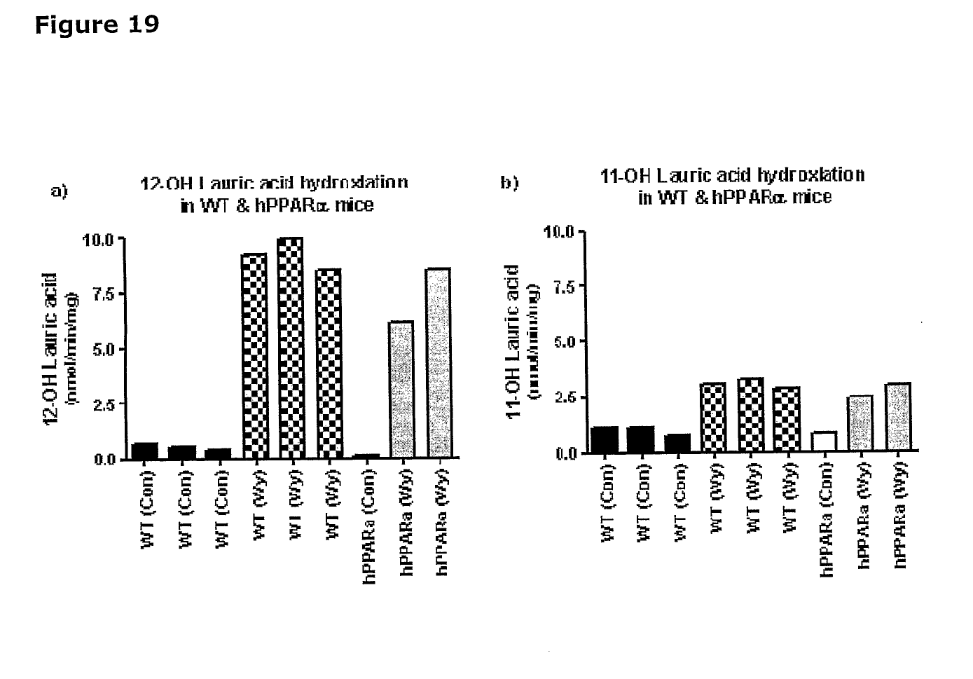

Liver microsomes from each available animal were evaluated for Cyp4a activity by measuring lauric acid hydroxylation (FIG. 19, Tables 17-18). Similar increases in formation of both metabolites (12-hydroxy; 12-OH and 11-hydroxy; 11-OH) were observed in both lines following treatment with Wy-14,64\tilde{3}. Interestingly, 12-OH lauric acid hydroxylation activity was lower in the vehicle-treated hPPARα mouse when compared to those seen in WT mice.

TABLE 17
Formation of 12-OH lauric acid in WT and hPPARα mice.

| | | | 12-OH | | | | | | |
|---|---|---|---|---|---|---|---|---|---|
| Mouse # | Mouse line | Treatment | (nmol/min/mg) | MEAN | SD | % MEAN | % SD | T-test | |
| 1 | WT | Control | 0.7 | 0.6 | 0.2 | 100 | 29 | | |
| 2 | WT | Control | 0.6 | | | | | | |
| 3 | WT | Control | 0.4 | | | | | | |
| 5 | WT | Wy-14,643 | 9.2 | 9.2 | 0.7 | 1669 | 129 | 0.002 | *** |
| 6 | WT | Wy-14,643 | 10.0 | | | | | | |
| 7 | WT | Wy-14,643 | 8.6 | | | | | | |
| 4 | hPPARα | Control | 0.2 | NA | NA | NA | NA | | |
| 8 | hPPARα | Wy-14,643 | 6.1 | NA | NA | NA | NA | NA | |
| 9 | hPPARα | Wy-14,643 | 8.5 | NA | NA | NA | NA | NA | |

Values are expressed as individual and/or mean ± SD (% mean own control ± % SD); where n = 3. A Student's t-test (2-sided) was performed on control vs_{:} Wy-14,643-treated WT mice, which revealed a statistically significant difference (*** p < 0.001).
NA; not applicable as n < 3.

TABLE 18
Formation of 11-OH lauric acid in WT and hPPARα mice.

| | | | 11-OH | | | | | | |
|---|---|---|---|---|---|---|---|---|---|
| Mouse # | Mouse line | Treatment | (nmol/min/mg) | MEAN | SD | % MEAN | % SD | T-test | |
| 1 | WT | Control | 1.1 | 1.0 | 0.2 | 100 | 23 | | |
| 2 | WT | Control | 1.1 | | | | | | |
| 3 | WT | Control | 0.7 | | | | | | |
| 5 | WT | Wy-14,643 | 3.0 | 3.0 | 0.2 | 308 | 20 | 0.001 | *** |
| 6 | WT | Wy-14,643 | 3.2 | | | | | | |
| 7 | WT | Wy-14,643 | 2.8 | | | | | | |
| 4 | hPPARα | Control | 0.8 | NA | NA | NA | NA | | |
| 8 | hPPARα | Wy-14,643 | 2.4 | NA | NA | NA | NA | NA | |
| 9 | hPPARα | Wy-14,643 | 3.0 | NA | NA | NA | NA | NA | |

Values are expressed as individual and/or mean ± SD (% mean own control ± % SD); where n = 3. A Student's t-test (2-sided) was performed on control vs_{:} Wy-14,643-treated WT mice, which revealed a statistically significant difference (*** p < 0.001).
NA; not applicable as n < 3.

Transcript Characterisation of hPPARα Mouse Model

RT-PCR was performed on liver samples from a single vehicle-treated WT mouse and a single hPPARα mouse) to assess whether the full-length human PPARα transcript was present in the hPPARα mouse.

Total RNA was prepared from one control WT and one hPPARα mouse liver tissue samples, and the RNA samples were purified using RNeasy kit (QIAGEN, Cat No. 74104). RT-PCR was conducted by using Superscript III One-Step RT-PCR Platinum Taq HiFi Kit (Invitrogen Corp. Cat. No. 12574-030), following the manufacturer's protocol. The products of RT-PCR were separated by electrophoresis on an agarose gel.

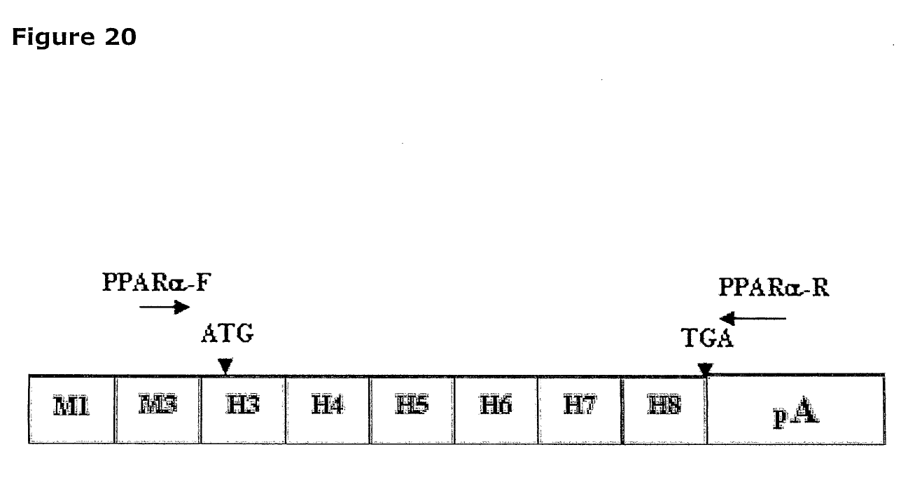

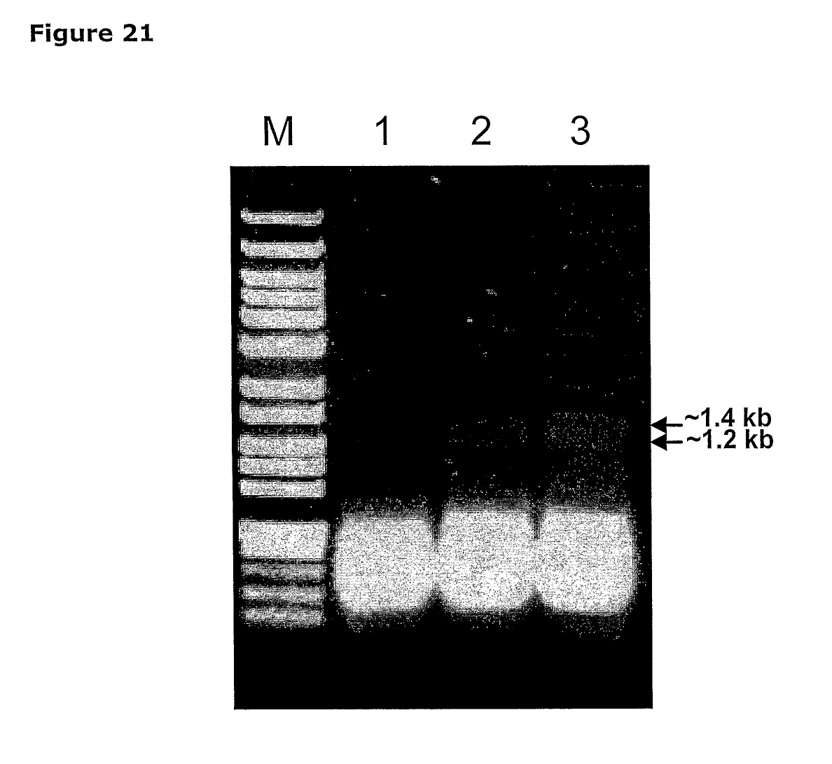

In order to confirm that the human PPARα transcript had a correct translation start site, primers termed PPARα-F and PPARα-R were used in the RT-PCR, as shown in FIG. 20. The expected product of RT-PCR, having a molecular weight of 1.4 kb, was detected (FIG. 21). The resulting cDNA was cloned and characterised by sequence analysis.

The targeting vector was designed in such a way that a chimeric construct of genomic and cDNA consisting of Exons 3-5, Intron 5, Exon 6, Intron 6 and Exon 7-8 of human PPARα was introduced into the mouse genome, replacing the coding region of Exon 3 of the murine PPARα gene. The construct was designed so that the transcript would be terminated by a polyA motif. The targeting vector carries an FRT-flanked neomycin resistance cassette, inserted into human Intron 5, which should be removed by FLP-mediated recombination to generate a humanised PPARα allele. Therefore, primers were designed to anneal to the construct's polyA region. The primers were as follows:

Forward primer:
PPARα_F cgc tct gtg gcc tgc ctg gcc ac
Reverse primer:
PPARα_R ccg cgc ctg gat ctc agg aat tcc

The specificity of the primers used was confirmed by the absence of a detectable band at the correct molecular weight indicating that, as expected, no human PPARα mRNA was present in the WT mouse analysed (FIG. 21). RT-PCR data demonstrated that at least two products (1.2 kb and 1.4 kb) could be generated from hPPARα mouse liver RNA, but not from WT mouse RNA. Although the expression of both fragments was low, subsequent cloning and sequencing analysis demonstrated three distinct transcripts, as described below.

RT-PCR was conducted using the Superscript III One-step RT-PCR Platinum Taq High Fidelity Kit (Invitrogen Corp, Cat #10574-030) according to the manufacturer's protocol. Total RNA (1 μg) was prepared from a vehicle-treated hPPARα mouse and mouse a vehicle-treated WT mouse and used directly for RT-PCR using primer pair PPARα-F and PPARα-R.

Duplicate 50 μl synthesis reactions were set up for each and run under the following conditions;

cDNA synthesis: 1 cycle of;

54° C. 30 min

94° C. 2 min

PCR amplification: 40 cycles of;

94° C. 20 sec

58° C. 30 sec

68° C. 2 min

Final extension: 1 cycle of;

68° C. 5 min

Restriction Analysis of Clones

A variant human PPARα mRNA species has been identified (Gervois et al, 1999). Sequence analysis revealed that the variant contained a 203 bp deletion and that the deleted fragment localized exactly at the boundaries of Exon 6, indicating that it is generated by an alternative splicing event skipping Exon 6. This resulted in a frame shift introducing a premature stop codon. The shorter transcript was predicted to result in the production of a truncated hPPARα protein lacking part of the hinge region and the entire ligand-binding domain. RNase protection analysis demonstrated that variant human PPARα mRNA was expressed in several human tissues and cells, representing between 20-50% of total PPARα mRNA. By contrast, variant PPARα could not be detected in rodent tissues. Thus, the PPARα variant transcript appeared to be specifically expressed in man.

In order to confirm that the 1.2 kb product observed following RT-PCR of hPPARα mouse liver RNA is the reported alternative spliced variant of human PPARα, the products of RT-PCR were separated by agarose gel electrophoresis using Qiagen gel purification kits. Fragments in the range of 1.2 to 1.4 kb were extracted from the gel, purified, ligated into the T/A site of the pCR4-TOPO vector using TOPO TA Cloning kit for Sequencing (Invitrogen Corp. Cat. no. K4575-01) and transformed into TOP10 ultracompetent cells. Colonies were screened by digestion with restriction enzyme BglII to determine the presence of an insert within the vector. One clone (clone #1) containing a 1.2 kb insert and six clones (clone #2, 6, 7, 9, 24 and 36) containing a 1.4 kb insert were sequenced. The ligated DNA was transformed into DH5α ultracompetent cells (Advantage, Dundee) and plated onto ampicillin plates to select for positive clones.

Sequence Analysis of Clones

Plasmid DNA for each positive clone was prepared using the Qiaprep Spin Miniprep kit according to the manufacturer's protocol (Qiagen; Cat #27160) and eluted in 50 μl elution buffer.

Approximately 300 ng of plasmid DNA was digested with BglII to release the insert from the vector backbones. The digests were loaded onto 1% agarose TAE gels.

Colonies resulting from the transformation plates were picked and added directly to a 20 μl PCR mix,

2 μl 10× buffer (NEB)

0.4 μl dNTPs (10 mM)

0.4 μl M13F sense primer (10 μM)

0.4 μl M13R antisense primer (10 μM)

0.1 μl Taq (NEB)

16.7 μl dH2O

and run under the following conditions;

1 cycle of: 95° C. 2 minutes;

40 cycles of: 95° C. 20 seconds; 56° C. 30 seconds

1 cycle of: 72° C. 100 seconds

Sequence analysis was performed by: Lark Technologies, Ltd., A Genaissance Company, Hope End, Takeley, Essex CM22 6TA. Alignments were performed using VectorNTI 8 Software, utilising Contig Express and Align-X modules and T-COFFEE alignment software (http://www.ch.embnet.org/software/TCoffee.html).

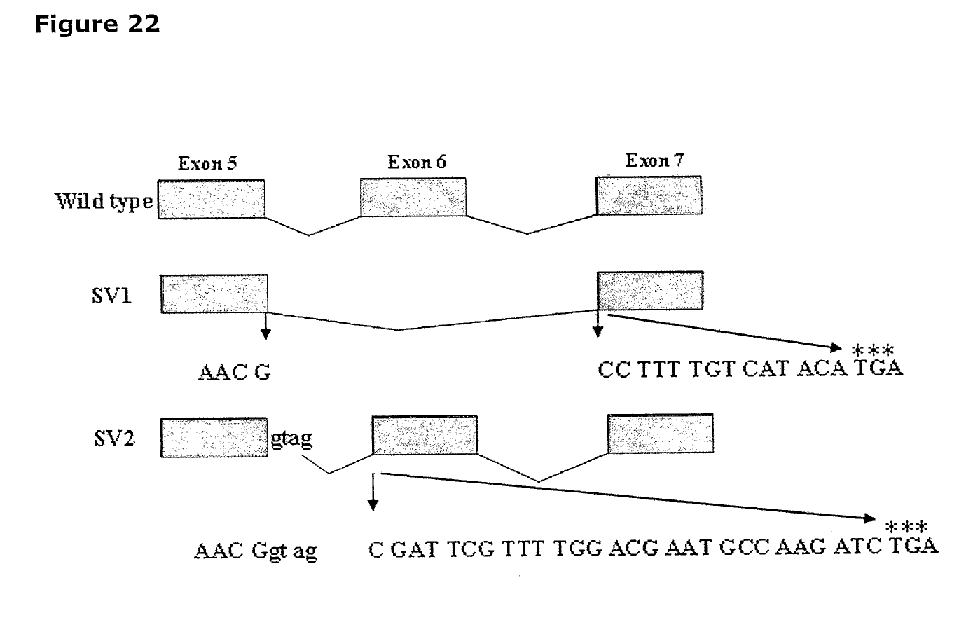

Sequence comparisons confirmed that the 1.2 kb insert in clone #1 was the spliced variant of human PPARα which lacked Exon 6 (SV1, as shown in FIG. 22). The 1.4 kb insert represents two types of transcripts, the normally spliced version (clone #2, 7, 9, and 24) and a splice variant SV2. Variant SV2 is a previously undescribed transcript using an alternative intronic 3′ splice site resulting in the addition of a 4 bp (GTAG) out-of-frame insertion into the 3′ end of Exon 5 and generation of a premature stop codon (FIG. 22). Based on the agarose gel electrophoresis, the ratio of 1.4 kb transcript to 1.2 kb transcript appeared to be about 1:1. However, the ratio of normally spliced transcripts to alternative spliced variants SV1 and SV2 could not be determined using the RT-PCR method.

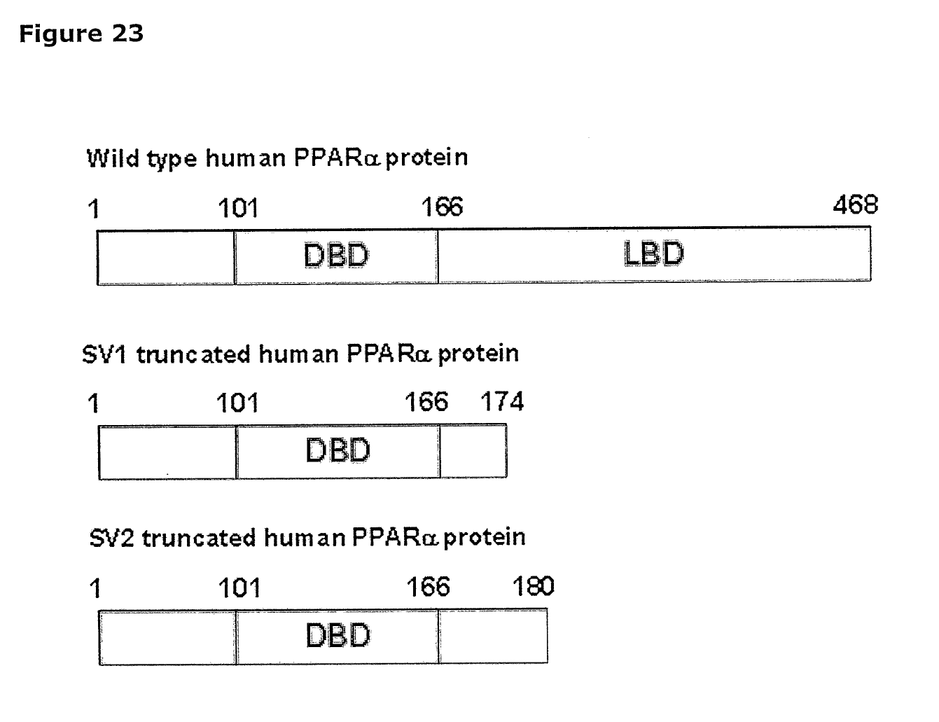

The alternatively spliced variants SV1 and SV2 encode truncated PPARα proteins containing 174 and 180 amino acids, respectively. Both truncated proteins lack a large region of the ligand binding domain (LBD), but they still contain the DNA binding domain (DBD) (FIG. 23). It remains to be determined whether the two alternative spliced transcripts can be transformed in hPPARα mice to form the truncated protein and whether they can bind to PPRE-containing DNA fragments. It has been reported that although truncated PPARα (PPARαtr) could not bind to PPRE elements in gel retardation assays, nuclear hPPARαtr was a potent repressor and could affect the transcriptional activity of full length hPPARα protein in vitro (Gervois et al, 1999).

In summary, a full-length human PPARα transcript specific to the humanised mouse was detected, cloned and verified by sequencing. Two alternatively spliced variants (SV1 and SV2) were identified in this study. Variant SV1 has been published and is a human specific alternatively spliced variant (Gervois et al, 1999). Variant SV2 is a new type of transcript with the addition of a 4 bp (GTAG) out-of-frame insert at the 3′ end of exon 5, resulting in a premature stop codon. The potential functions of truncated PPARα protein in humanised mice are not known.

TaqMan® Analysis of mPPARα and hPPARα mRNA Expression

Mouse liver samples were snap frozen in liquid nitrogen to preserve RNA integrity. Taqman® quantitative RT-PCR RNA isolation was carried out using the RNeasy mini kit from a vehicle-treated hPPARα mouse and a vehicle-treated WT mouse.

Primers specific for murine PPARα and human PPARα (Assay-on-demand Cat #, Mm00440939_m1 and Hs00947539_m1, Applied Biosystems, respectively) were employed for TaqMan® analysis of PPARα expression in mouse liver from WT and hPPARα mice. The murine PPARα primers were designed to anneal between Exons 7 and 8 of the murine PPARα sequence and the human PPARα primers were designed to amplify Exons 7 to 9 of the human sequence.

Levels of murine and human PPARα mRNA were quantified by quantative RT-PCR (TaqMan®) in the site of normal PPARα expression (liver) in WT and hPPARα mice (Table 19). RNA was extracted from mouse liver. cDNA was synthesised from all available RNA samples, and TaqMan® analysis was performed in all available samples using primers specific of murine PPARα and human PPARα (Assay-on-demand kit Applied Biosystems). Murine α-actin was used as the internal standard (Assay-on-demand kit Cat #43S2933E, Applied Biosystems).

Human PPARα transcripts were found in hPPARα mice, but not in WT mice. Murine PPARα was identified in WT mice but not in the hPPARα mice. The level of PPARα mRNA detected was similar in both models.

TABLE 19
TaqMan® analysis of PPARα mRNA in mouse liver. Total RNA was isolated
from livers of WT and hPPARα mice. Subsequent cDNA synthesis and qRT-PCR was
performed with specific primers for murine or human PPARα. Values represent cycling
time (CT) values for individual mice. The internal standard used for each assay was
murine α-actin. Grey boxes denote CT values <35 = no mRNA expression. The lower
the CT value, the greater the level of mRNA expression.

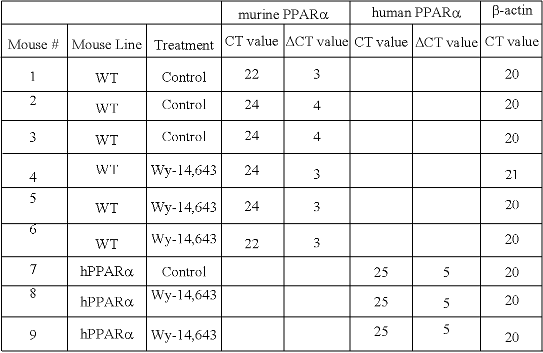

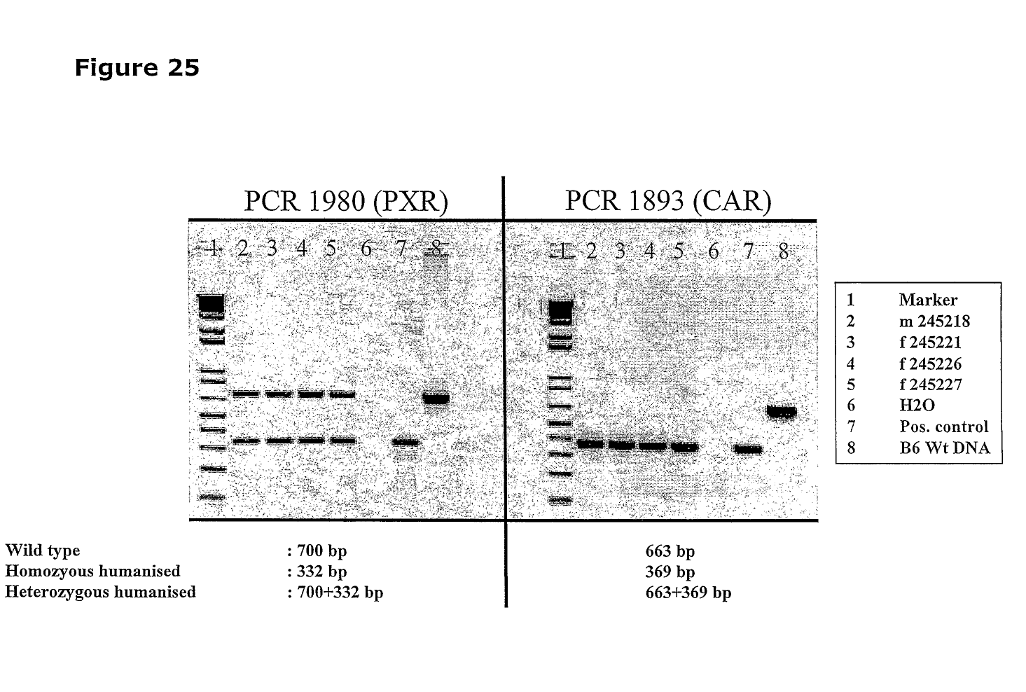

Mice have now been generated that are double homozygous for human CAR and PPARα and heterozygous for human PXR. FIG. 25 shows PCR confirmation of these double homozygous CAR/PPARα and heterozygous PXR humanised mice. Mice with the IDs m 245218, f 245221, f 245226 and f 245227 are homozygous humanised for CAR and heterozygous humanised for PXR. Triple humanised PXR/CAR/PPARα mice will be available very shortly, following protocols set up in existing breeding programs at TaconicArtemis.

### Example 5

### huAhR and koAhR

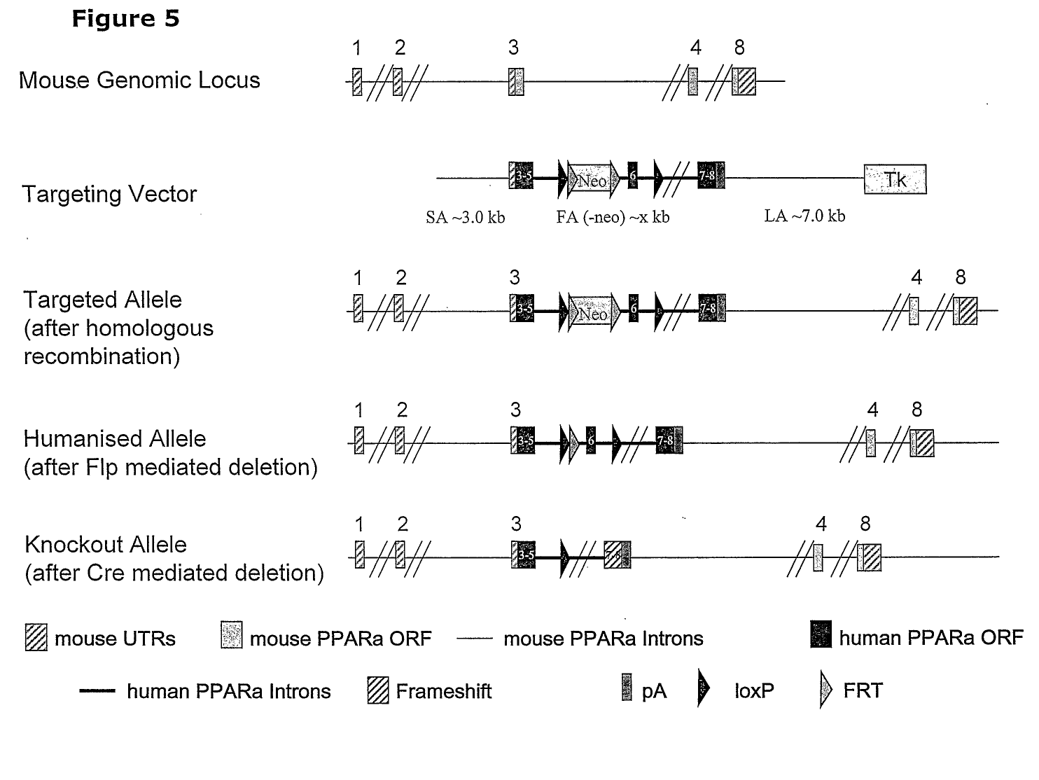

A DNA sequence encoding human AhR has been inserted into the mouse AhR locus (knock-in) as shown in FIG. 5, enabling expression of human AhR under the control of the mouse AhR promoter. The DNA sequence encoding human AhR comprises exons 3-11 of the human AhR gene (FIG. 5). The targeting vector(s) include(s) sequence elements that enable Cre-mediated AhR knock-out to produce koAhR (FIG. 5).

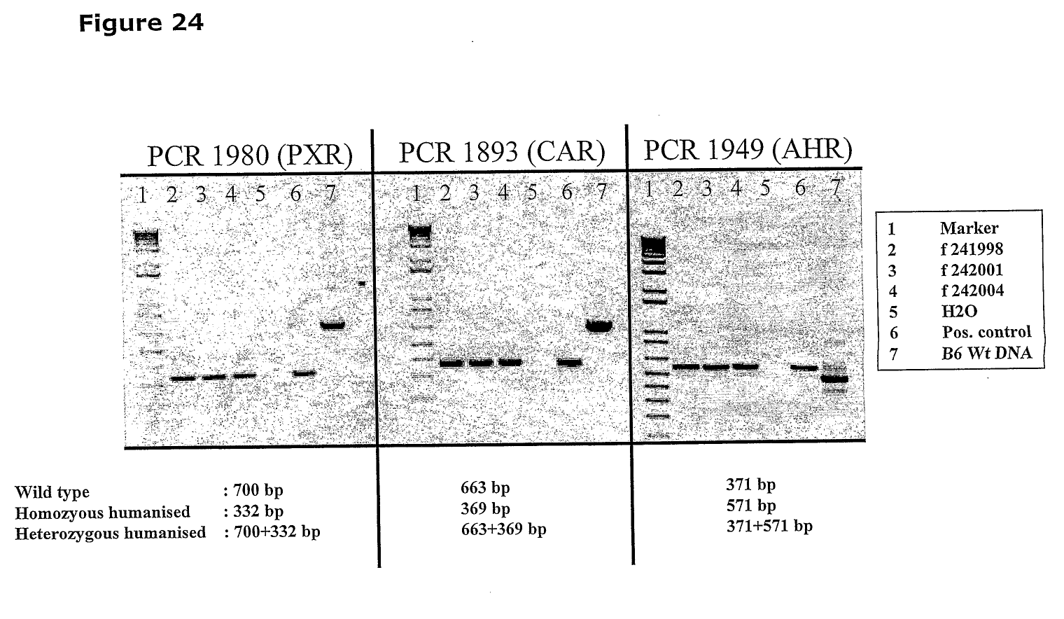

Mice that are triple homozygous humanised for PXR, CAR and AHR have now been generated. FIG. 24 shows PCR confirmation of triple homozygous PXR/CAR/AhR humanised mice. Mice with the IDs f 241998, f 242001 and f 242004 are triple homozygous humanised for PXR, CAR and AHR. Such mice will of extreme value in the assays described herein.

### Example 6

### Proof of Concept: the Effect of the Non-Genotoxic Carcinogen, PB, in Rodents

Phenobarbital (hereafter PB) is known to cause liver cancer in mice and rats but does not do the same in humans. Following long term treatment to PB, rodents develop liver tumours. Initially, PB causes a hyperplastic response and cell replication and liver weight increases for the first two weeks of treatment. However, after approximately two years, liver tumours become evident in treated animals. Under this type of analysis, PB would be deemed unsafe for use in humans. However, PB is indeed safe, having been sold for many years with no record of liver tumour incidence in treated patients. This illustrates the shortcomings of current animal models to test for drug safety in humans, and means that there is unnecessary drug attrition occurring at this stage of the safety testing process. The question which drug companies need to answer, at as early a stage of testing as possible, is whether hyperplastic responses to chemicals observed in animals are actually relevant to man?

The transcription factor CAR is known to be essential for responses to PB-like inducers. Wei et al, 2000 (Nature) showed that in wild type mice, CAR activators increased liver mass, reflective of cellular hypertrophy and hyperplastic response. In contrast, CAR KO mice showed no increased liver mass after treatment with CAR activators. Furthermore, induction of DNA synthesis as determined by increased incorporation of BrdU in wild type mice was also absent in CAR KO mice. Similarly, Cheung et al, 2004 showed that humanisation of the mice for PPARα decreased the increase in liver weight elicited by treatment with various drugs in wild type animals. The humanised mice also showed a lack of increased replicative DNA synthesis, as seen in the wild type animals.

In order to assess whether humanised PXR/CAR mice mimic the response to PB in humans, the huPXR/huCAR and PXRKO/CARKO mouse models were used. The mutant mouse strains were obtained from Artemis. The WT mouse strain C57BL/6J was obtained from Harlan (UK). All animals were between 10 and 16 weeks of age.The following parameters were studied:
Liver/body weight ratios
BrdU incorporation analysed as a measure of cell proliferation
Haematoxylin and eosin (H&E) liver histopathology
Expression and activity of P450s by SDS-PAGE and Western blotting in liver microsomes
Apoptotic indices as analysed by TUNEL assay in the liver

The study consisted of 6 groups with 10 WT mice per group, 9-10 PXRKO/CARKO mice per group and 9 huPXR/huCAR mice per group (Table 19). All animals were implanted with osmotic pumps (Alzet 2001) containing bromodeoxyuridine (BrdU, 15 mg/ml in phosphate buffered saline [PBS], pH7.4) 5 days before termination for all mice (Day −1). Post operation all animals had no abnormalities detected. On Day 1 all animals were dosed with either 80 mg/kg PB/saline or saline alone by intraperitoneal injection for 4 days as detailed in Table 19.

TABLE 19
Experimental design

| | | Number | | | | | Implant | | |
|---|---|---|---|---|---|---|---|---|---|
| | | mice/ | Mouse | Inducing | Dose | | minipumps | First dose | Termination |
| Grp | Mouse # | group | Strain | agent | (mg/kg) | Regimen | with Brdu | date | Date |
| 1 | 11-10 | 10 | WT | Control | — | 4 x daily | 17-Oct-07 | 18-Oct-07 | 22-Oct-07 |
| 2 | 11-19 | 9 | huPXR/ | Control | — | 4 x daily | 17-Oct-07 | 18-Oct-07 | 22-Oct-07 |
| | | | huCAR | | | | | | |
| 3 | 20-28 | 9 | PXRKO/ | Control | — | 4 x daily | 17-Oct-07 | 18-Oct-07 | 22-Oct-07 |
| | | | CARKO | | | | | | |
| 4 | 29-38 | 10 | WT | PB | 80 | 4 x daily | 17-Oct-07 | 18-Oct-07 | 22-Oct-07 |
| 5 | 39-47 | 9 | huPXR/ | PB | 80 | 4 x daily | 17-Oct-07 | 18-Oct-07 | 22-Oct-07 |
| | | | huCAR | | | | | | |
| 6 | 48-57 | 10 | PXRKO/ | PB | 80 | 4 x daily | 17-Oct-07 | 18-Oct-07 | 22-Oct-07 |
| | | | CARKO | | | | | | |

PXR/CAR-Dependent Hepatomegaly by PB

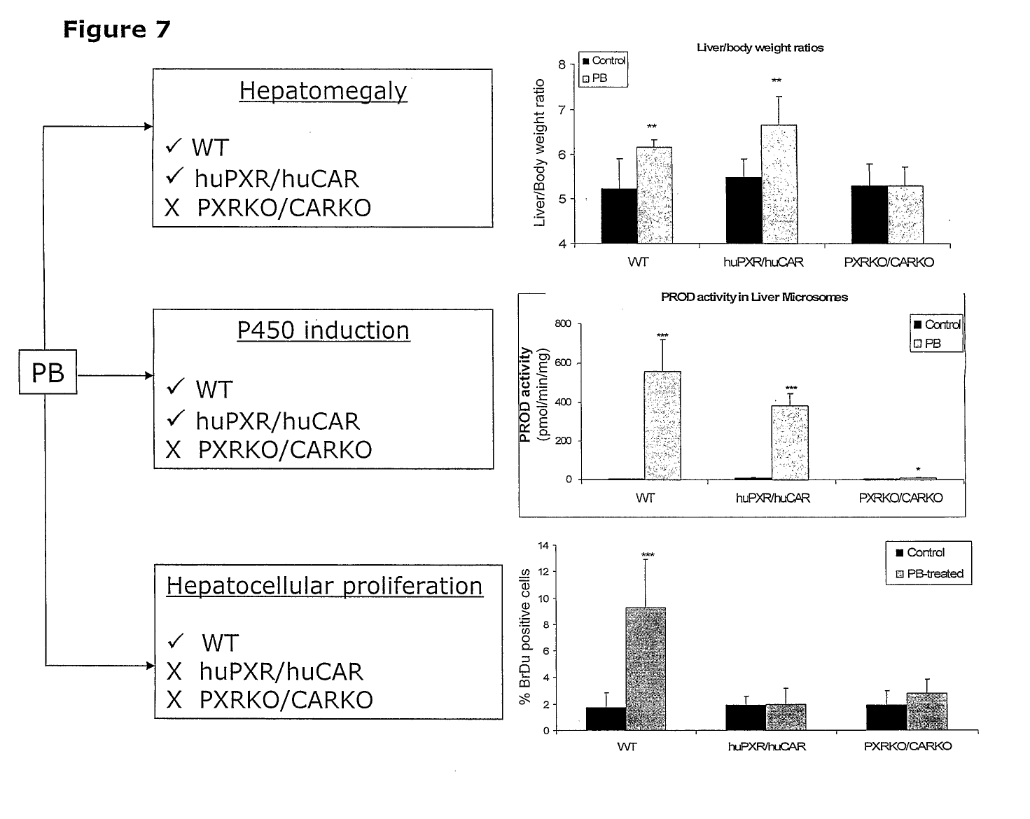

Following treatment with PB or the vehicle, the mice were sacrificed and their livers were removed and weighed. Hepatomegaly was observed in both the WT and “humanised” mice, but not in the PXRKO/CARKO in response to PB treatment, as shown by increases in liver body weight ratios of 118% and 122%, respectively (Table 20, FIG. 7).

TABLE 3
Liver/body weight ratios

| | | Body | Body | | Liver/Body |
|---|---|---|---|---|---|
| Mouse | | weight (g) - | weight (g) - | Liver | weight |
| line | Treatment | Day 1 | Day 5 | weight (g) | Ratio |
| WT | Control | 27.9 ± 2.1 | 27.7 ± 2.4 | 1.44 ± 0.22 | 5.2 ± 0.7 |
| | | (100 + 8) | (100 ± 8.7) | (100 ± 15) | (100 ± 13.5) |
| | PB | 26.4 ± 1.8 | 23.2 ± 1.3 * | 1.44 ± 0.09 | 6.2 ± 0.2 ** |
| | | (106 + 6) | (84 ± 5) | (101 ± 6) | (118 ± 3) |
| huPXR/huCAR | Control | 25.4 ± 1.9 | 25.6 ± 1.9 | 1.40 + 0.14 | 5.5 ± 0.4 |
| | | (100 ± 7) | (100 ± 8) | (100 ± 10) | (100 ± 8) |
| | PB | 26.3 ± 1.2 | 25.4 ± 1.5 | 1.69 ± 0.16 ** | 6.7 ± 0.6 ** |
| | | (104 ± 5) | (99 ± 6) | (121 ± 11) | (122 ± 12) |
| PXRKO/CARKO | Control | 27.2 + 1.9 | 27.3 ± 2.4 | 1.45 ± 0.22 | 5.3 ± 0.5 |
| | | (100 ± 7) | (100 ± 9) | (100 ± 15) | (100 ± 10) |
| | PB | 27.2 + 2.2 | 26.6 + 2.2 | 1.42 + 0.23 | 5.3 ± 0.4 |
| | | (100 ± 8) | (98 ± 8) | (98 ± 16) | (100 ± 9) |

Values are expressed as Mean ± SD (% mean own control ± % SD); n = 3. A Student's t-test (2-sided) was performed on the results; * and ** statistically different from control mice at p < 0.05 and p < 0.01, respectively.

Hepatic Cell Proliferation

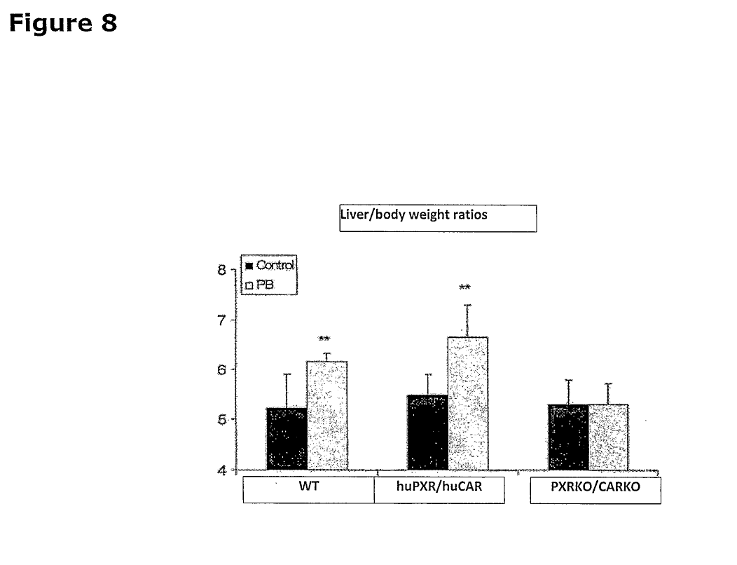

All mouse liver and duodenum sections were analysed for BrdU incorporation as a measure of cell proliferation. The method used was an indirect BrdU labelling assay. PB increased the hepatocellular labelling index (S-phase) in the WT mice by approximately 5-fold and appeared to have no effect on cell proliferation in the huPXR/huCAR or PXRKO/CARKO (FIG. 8, Table 21).

Liver in situ Cell Death

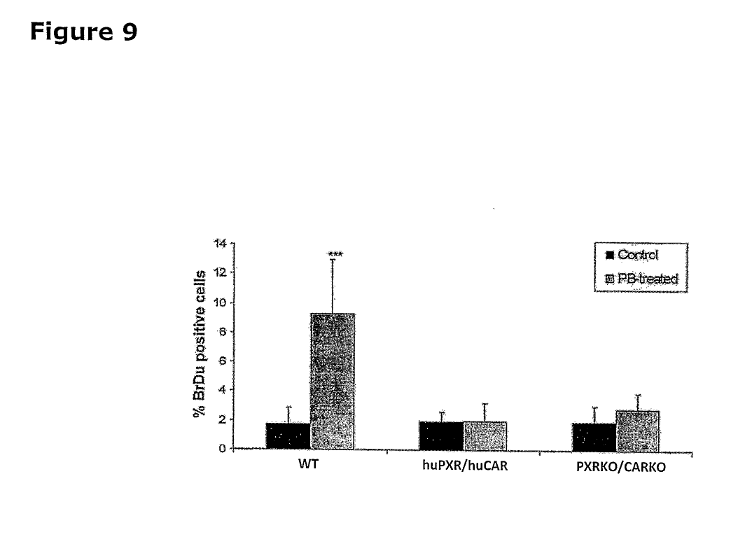

A 50% inhibition of hepatocellular apoptosis by non-genotoxic carcinogens has been previously demonstrated with consistency in rats. However, no such consistency has been observed in mice. An indirect TUNEL labelling assay was used to analyse hepatic in situ death (FIG. 9/Table 21). The present study has shown a marker variation in apoptotic indices in mouse liver. Thus, small (e.g. 50%) compound-induced decreases, upon a low background, were not readily demonstrable.

H&E Analysis

Two samples of the liver (one from the lobe, one from the median lobe) and one sample of the small intestine were taken and preserved in 4% neutral buffered formaldehyde (NBF). The preserved liver samples of all mice of all groups were trimmed, processed and embedded in paraffin. The paraffin-embedded samples were sent to Progenix, Inverkeithing, UK where they were sectioned at a nominal thickness of about 5 \acute{æ}m and then stained with haematoxylin and eosin (H&E). One section of each organ sample was examined by Dr. Ortwin Vogel, Consultant Pathologist, Kiel, Germany. Subsequent to his histopatholoical analysis of all H&E stained mouse livers and small intestines, Dr. Vogel reported the following finding;

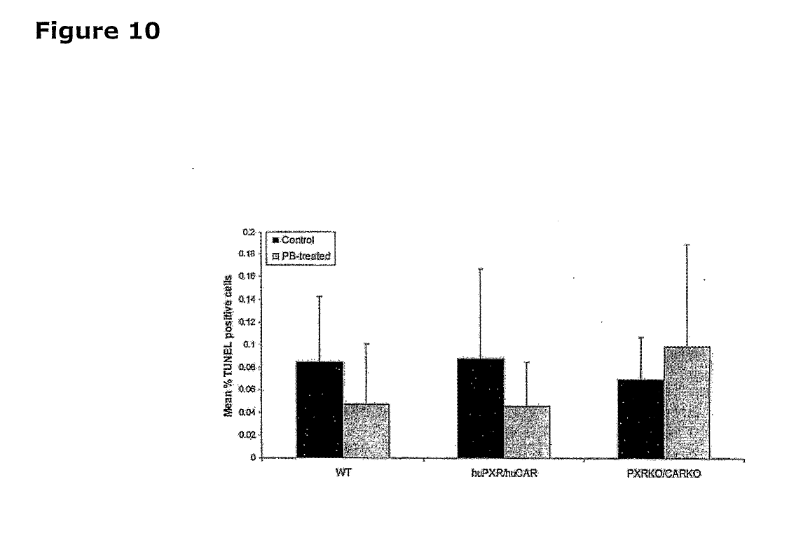

Microscopically, a slight to moderate centrilobular hepatocellular hyperthrophy was noted, in PB treated huPXR/huCAR and WT animals (FIG. 10). This finding is considered to be related to the treatment with PB. In contrast, no unequivocal evidence of a hepatocellular hypertrophy was noted in PXRKO/CARKO mice following treatment with PB. In addition, mitotic figures indicating hepatocellular proliferation were noted mainly in PB-treated WT mice but also, with a lower incidence, in the “humanised” and null mouse lines.

TABLE 21
Hepatic S-phase Labelling Indices in PB-treated mice
% BrDu positive

| Mouse # | Mouse line | Treatment | Cells | Mean | SD | % mean | % SD | T-Test |
|---|---|---|---|---|---|---|---|---|
| 1 | WT | Control | 4.39 | 1.75 | 1.10 | 100 | 63 | |
| 2 | WT | Control | 2.14 | | | | | |
| 3 | WT | Control | 2.18 | | | | | |
| 4 | WT | Control | 2.35 | | | | | |
| 5 | WT | Control | 1.39 | | | | | |
| 6 | WT | Control | 1.31 | | | | | |
| 7 | WT | Control | 0.74 | | | | | |
| 8 | WT | Control | 0.99 | | | | | |
| 9 | WT | Control | 0.77 | | | | | |
| 10 | WT | Control | 1.24 | | | | | |
| 11 | huPXR/huCAR | Control | 1.07 | 1.93 | 0.67 | 100 | 35 | |
| 12 | huPXR/huCAR | Control | 1.65 | | | | | |
| 13 | huPXR/huCAR | Control | 2.02 | | | | | |
| 14 | huPXR/huCAR | Control | 1.53 | | | | | |
| 15 | huPXR/huCAR | Control | 1.18 | | | | | |
| 16 | huPXR/huCAR | Control | 2.02 | | | | | |
| 17 | huPXR/huCAR | Control | 3.05 | | | | | |
| 18 | huPXR/huCAR | Control | 2.79 | | | | | |
| 19 | huPXR/huCAR | Control | 2.03 | | | | | |
| 20 | PXRKO/CARKO | Control | 2.75 | 1.96 | 1.04 | 100 | 53 | |
| 21 | PXRKO/CARKO | Control | 3.86 | | | | | |
| 22 | PXRKO/CARKO | Control | 0.74 | | | | | |
| 23 | PXRKO/CARKO | Control | 1.8G | | | | | |
| 24 | PXRKO/CARKO | Control | 1.28 | | | | | |
| 25 | PXRKO/CARKO | Control | 1.09 | | | | | |
| 26 | PXRKO/CARKO | Control | 1.10 | | | | | |
| 27 | PXRKO/CARKO | Control | 2.02 | | | | | |
| 28 | PXRKO/CARKO | Control | 2.94 | | | | | |
| 29 | WT | PB | 9.45 | 9.30 | 3.64 | 531 | 208 | 0.0002 |
| 30 | WT | PB | 5.54 | | | | | |
| 31 | WT | PB | 16.69 | | | | | |
| 32 | WT | PB | NS | | | | | |
| 33 | WT | PB | 6.S1 | | | | | |
| 34 | WT | PB | 6.32 | | | | | |
| 35 | WT | PB | 9.50 | | | | | |
| 36 | WT | PB | 8.09 | | | | | |
| 37 | WT | PB | 7.64 | | | | | |
| 38 | WT | PB | 13.60 | | | | | |
| 39 | huPXR/huCAR | PB | 0.37 | 2.00 | 1.18 | 104 | 61 | 0.87 |
| 40 | huPXR/huCAR | PB | 0.-18 | | | | | |
| 41 | huPXR/huCAR | PB | 1.19 | | | | | |
| 42 | huPXR/huCAR | PB | 1.86 | | | | | |
| 43 | huPXR/huCAR | PB | 3.GO | | | | | |
| 44 | huPXR/huCAR | PB | 1.98 | | | | | |
| 45 | huPXR/huCAR | PB | 3.18 | | | | | |
| 46 | huPXR/huCAR | PB | 3.24 | | | | | |
| 47 | huPXR/huCAR | PB | 2.13 | | | | | |
| 48 | PXRKO/CARKO | PB | 2.16 | 2.83 | 1.07 | 144 | 55 | 0.09 |
| 49 | PXRKO/CARKO | PB | 1.98 | | | | | |
| 50 | PXRKO/CARKO | PB | 1.82 | | | | | |
| 51 | PXRKO/CARKO | PB | 2.30 | | | | | |
| 52 | PXRKO/CARKO | PB | 3.77 | | | | | |
| 53 | PXRKO/CARKO | PB | 5.10 | | | | | |
| 54 | PXRKO/CARKO | PB | 3.71 | | | | | |
| 55 | PXRKO/CARKO | PB | 1.88 | | | | | |
| 56 | PXRKO/CARKO | PB | 3.06 | | | | | |
| 57 | PXRKO/CARKO | PB | 2.51 | | | | | |

Values are expressed as Mean ± SD. A Student's t-test (2-sided) was performed on the results; with *** statistically different from control mice at pO.OOl. NS, no sample available for analysis

All other microscopic findings recorded in the liver did not significantly distinguish PB-treated mice from control mice or the differences were regarded as random events. All these findings are considered to be spontaneous in nature and within the normal background pathology commonly seen in mice. No microscopic findings were recorded in the small intestine. Under the conditions of this study, PB (80 mg/kg/4 days/IP) produced pathological evidence of a hepatocellular hypertrophy/hyperplasia in mice of the WT strain as well as in mice of the huPXR/huCAR strain. Evidence of hepatocellular hyperplasia occurred in PB-treated mice of the PXRKO/CARKO strain.

Hepatic P450 Induction

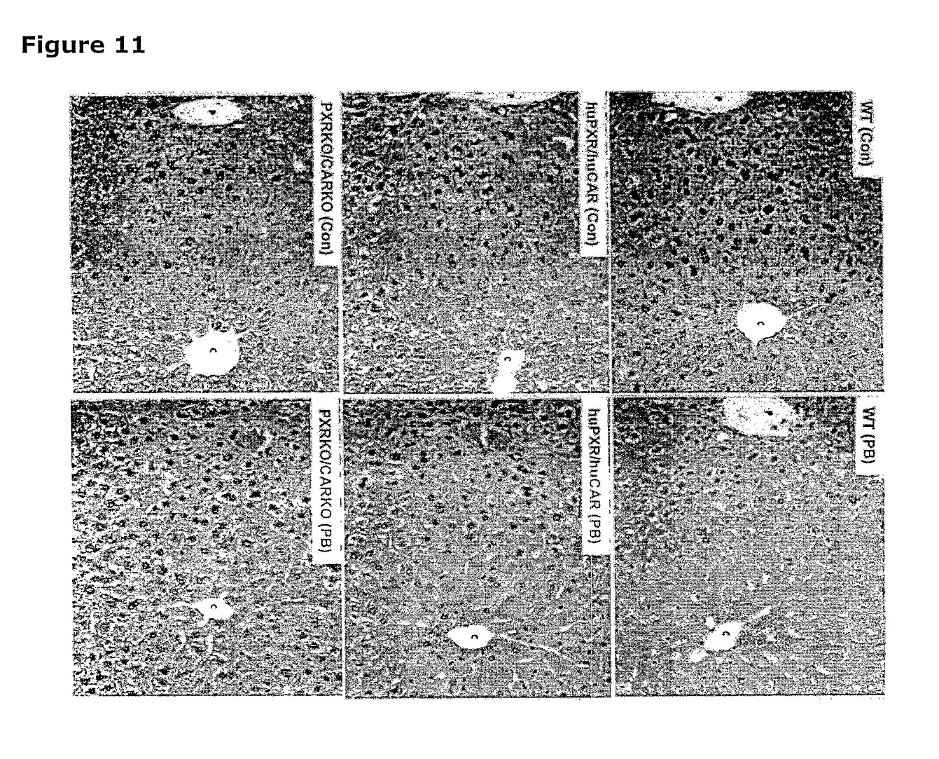

P450 catalytic activities in the liver microsomal fractions were quantified. For the quantification of mouse Cyp2b10 and Cyp3a11 activities, the dealkylation of pentoxyresofurin (PROD) and the debenzylation of benzyloxyquinoline (BQ) activities, respectively. In addition, benzyloxyresorufin-O-demethylase (BROD), methoxyresorufin-O-demethylase (MROD) and 7-ethoxyresorufin-)-deethylase (EROD) activities were also measured and evaluated (see FIG. 11).

EROD activity is indicative of Cyp1a1/1a2 and Cyp1b1 in the mouse, whereas MROD is a substrate for Cyp1a2. However, other isoforms may be contributing to the measured activities. The results from the two enzyme assays are in reasonably good agreement with each other (FIG. 11a-b) As the Cyp1a2 gene is constitutively expressed, this may explain the basal levels of activity observed in both activity assays. Cyp1a1 is only expressed following induction in mice (Ikeya et al, 1989) and Phenobarbital has been reported not to induce this P450 in C57BL/6J mice (Sakuma et al, 1999). The MROD and EROD data demonstrated that enzyme activities were increased by PB treatment in WT and huPXR/huCAR mice. Both assays revealed no increase due to PB treatment in the PXRKO/CARKO line. Earlier data using the PXRICAR mouse panel demonstrated PB, at 40 mg/kg/4 days, could activate Cyo1a2 via the CAR mediated pathway, although other data are consistent with these earlier findings.

A 5-fold increase in BQ activity was seen in the PB-treated huPXR/huCAR mice, whereas a marginal increase was detected in the WT animals (FIG. 11c). These data demonstrate a clear species difference between the mouse lines, indicating that the human receptors have a greater sensitivity to PB than their murine counterparts.

Furthermore, this mechanism appears to be dependent on the presence of the receptors, as verified by the absence of Cyp3a11 induction in the PB-treated PXRKO/CARKO mice.

BROD and PROD are markers of Cyp2b10 activity in the mouse. In both the WT and huPXR/huCAR mouse lines, marker Cyp2b10 induction was observed, at similar levels following treatment with PB (FIG. 11d-e). However, PROD or BROD activities were not altered in the PXRKO/CARKO mice upon exposure to PB. Overall, PB induced P450 catalytic activities in the WT and huPXR/huCAR mouse lines, but not in the animals which were devoid of these receptors. This clearly indicates that PB-mediated P450 induction is CAR/PXR-dependent.

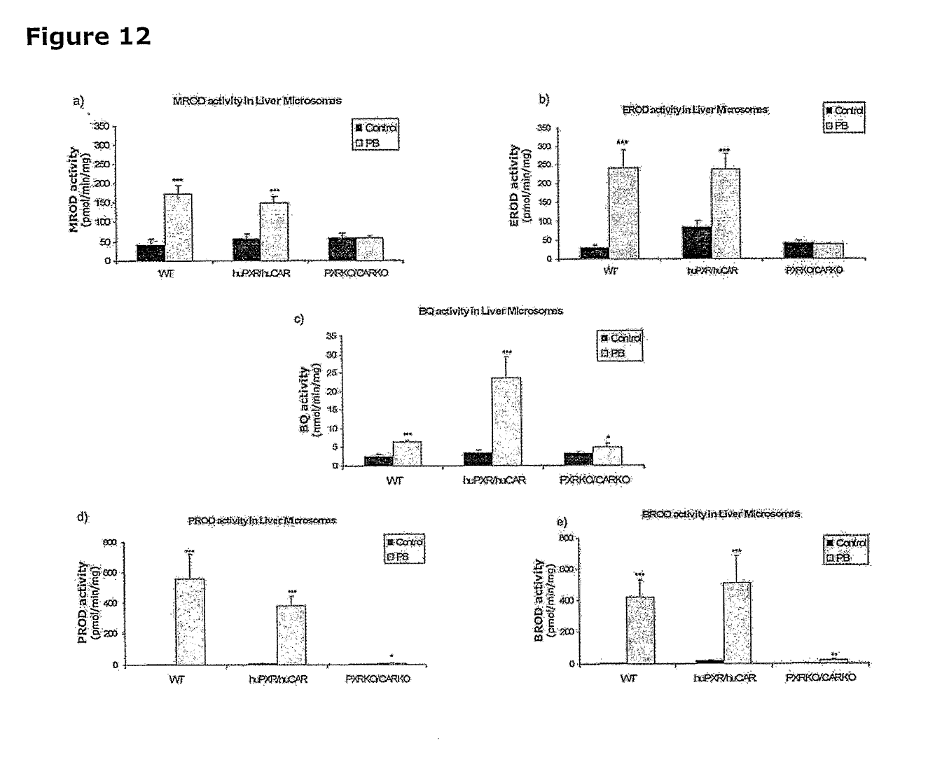

In accordance with P450 activity data, quantification of Cyp2b10 and Cyp3a11 protein in pooled mouse liver microsomes by Western blotting revealed that both P450s were induced by PB in WT and humanised mouse lines but not in the PXRKO/CARKO animals (FIG. 12). Furthermore, the species difference in Cyp3a11 induction is confirmed at the protein levels, suggesting that PB has a greater sensitivity for the human receptors over its murine equivalents.

In conclusion it can be said that hepatomegaly occurred only in WT mice and in huPXR/huCAR mice. The KO PXR/KO CAR showed no effect. The same pattern was mirrored for P450 induction. However, the most striking result was found when the mice were tested for hepatocellular proliferation (as determined by incorporation of the DNA precursor BrdU), where it was found that only the WT mice displayed a proliferation of hepatocytes. Both KO and humanised animals showed no proliferative effect whatsoever.

Important conclusions, therefore, are that:
PB induced hypertrophy and hyperplasia in WT mice.
PB did not induce hepatomegaly (neither hypertrophy or hyperplasia) in PXRKO/CARKO mice.
PB induced hypertrophy but not hyperplasia in huPXR/huCAR mice.

These mice and huPXR/huCAR/huPPARα will be of considerable value in assessing the true hazard of non-genotoxic rodent “liver growth carcinogens” to humans. They will also provide useful tools to unravel the complexities of xenobiotic-induced liver growth and species differences in such growth.

### REFERENCES

Cattley R C, DeLuca J, Elcombe C R, Fenner-Crisp P, Lake B G, Marsman D S, Pastoor T A, Popp J A, Robinson D E, Schwetz B, Tugwood J, Wahli W (1998). Do peroxisome proliferating compounds pose a hepatocarcinogenic hazard to humans? Regul Toxicol Pharmacol. 27:47-60.

Cattley R C (2004). Peroxisome proliferators and receptor-mediated hepatic carcinogenesis. Toxicol Pathol. 32 Suppl 2:6-11.

Cheung C, Akiyama T E, Ward J M, Nicol C J, Feigenbaum L, Vinson C, Gonzalez F J (2004). Diminished hepatocellular proliferation in mice humanized for the nuclear receptor peroxisome proliferator-activated receptor alpha. Cancer Res. 64:3849-54.

Gervois P, Torra I P, Chinetti G, Grötzinger T, Dubois G, Fruchart J C, Fruchart-Najib J, Leitersdorf E, Staels B (1999). A truncated human peroxisome proliferator-activated receptor alpha splice variant with dominant negative activity. Mol Endocrinol. 13:1535-49.

Gonzalez F J, Shah Y M (2008). PPARalpha: mechanism of species differences and hepatocarcinogenesis of peroxisome proliferators. Toxicology 246:2-8.

Graham M J, Lake B G (2008). Induction of drug metabolism: species differences and toxicological relevance. Toxicology 254(3):184-91

Ikeya, K., Jaiswal, A. K., Owens, R. A., Jones, J. E., Nebert, D. W. & S. Kimura (1989) Human CYP1A2: sequence, gene structure, comparison with the mouse and rat orthologous gene, and differences in liver CYP1A2 mRNA expression. Mol. Endocrinol. 3: 1399-1408

Macdonald N, Holden P R, Roberts R A (1999). Addition of peroxisome proliferator-activated receptor alpha to guinea pig hepatocytes confers increased responsiveness to peroxisome proliferators. Cancer Res. 59:4776-80.

Morimura K, Cheung C, Ward J M, Reddy J K, Gonzalez F J (2006). Differential susceptibility of mice humanized for peroxisome proliferator-activated receptor alpha to Wy-14,643-induced liver tumorigenesis. Carcinogenesis. 27:1074-80.

Sakuma, T., Ohtake, M., Katsurayama, Y., Jarukamjorn, K. & N. Nemoto (1999) Induction of CYP1A2 by phenobarbital in the livers of Aryl hydrocarbon-responsive and -nonresponsive mice. Drug Metab. Dispos. 27: 379-384.

Shearer B G, Hoekstra W J (2003). Recent advances in peroxisome proliferator-activated receptor science. Curr Med Chem. 10:267-80.

Yang Q, Nagano T, Shah Y, Cheung C, Ito S, Gonzalez F J (2008). The PPAR alpha-humanized mouse: a model to investigate species differences in liver toxicity mediated by PPAR alpha. Toxicol Sci. 101:132-9.

1. A method for screening a non-genotoxic carcinogen for safety in humans, the method comprising exposing a preparation of cells to the non-genotoxic carcinogen and monitoring for a physiological effect, wherein the animal is humanised for at least two nuclear transcription factors selected from the group consisting of PXR, CAR, PPARα and AHR and wherein the endogenous equivalent genes in the animal have been rendered inoperable.

2. A method according to claim 1, wherein the animal is humanised for at least PXR and CAR; PXR and PPARα; CAR and PPARα; PXR and AHR; PPARα and AHR; or CAR and AHR.

3. A method according to claim 1, wherein said animal is a transgenic non-human animal which has been humanised for the nuclear transcription factors CAR, PXR and PPARα, and in which the endogenous equivalent genes have been rendered inoperable.

4. A method according to claim 3, wherein said animal has been additionally humanised for the AHR receptor, and in which the endogenous equivalent gene has been rendered inoperable.

5. A method according to claim 1, wherein said animal is a transgenic non-human animal which has been humanised for the nuclear transcription factors CAR, PXR and AHR, and in which the endogenous equivalent genes have been rendered inoperable.

6. A method according to claim 1, wherein all endogenous equivalent genes of said animal have been rendered inoperable in all tissues.

7. A method according to claim 1 in which the expression level of the genes rendered inoperable in said animal is less than 10% of the wild type expression level.

8. A method according to claim 1, wherein the human transcription factor gene sequences are inserted at the point in the host animal chromosome where the endogenous equivalent target genes naturally occur.

9. A method according to claim 1, wherein the human transcription factor genes are inserted at the point in the host animal chromosome where the endogenous equivalent target genes naturally occur, replacing the endogenous equivalent target genes in the host chromosomes.

10. A method according to claim 1, wherein transcription of said human transcription factor genes in said animal is under the control of one or more endogenous regulatory sequence(s) of the host animal.

11. A method according to claim 1, wherein transcription of said human replacement gene sequence in said animal is under the control of the endogenous human regulatory sequence(s).

12. A method according to claim 1, wherein said animal has additionally been humanised for one, two or all three of CYP3A4, CYP2C9, and CYP2D6.

13. A method according to claim 1, wherein said animal has additionally been humanised for MDR and/or MRP.

14. A method according to claim 1, wherein said animal has additionally been humanised for UGT1A.

15. A method according to claim 1, wherein said animal is a rodent, more preferably, a mouse.

16. A method according to claim 1, used for efficacy screening, PK/PD modelling or drug safety testing.

17. A method according to claim 1, wherein the non-genotoxic carcinogen is a ligand for at least one of PXR, CAR or PPARα.

18. A method according to claim 1, wherein said physiological effect is metabolism of the non-genotoxic carcinogen.

19. A method according to claim 1, wherein said physiological effect is hepatomegaly, P450 induction or hepatocellular proliferation.

20. A method for screening a non-genotoxic carcinogen for safety in humans, the method comprising exposing a preparation of cells to the non-genotoxic carcinogen in vitro and monitoring for a physiological effect, wherein the cell is derived from an animal humanised for at least two nuclear transcription factors selected from the group consisting of PXR, CAR, PPARα and AHR and wherein the endogenous equivalent genes in the animal have been rendered inoperable.

21. A transgenic non-human animal which has been humanised for the nuclear transcription factors CAR, PXR and PPARα, and in which the endogenous equivalent genes have been rendered inoperable.

22. A transgenic non-human animal which has been humanised for the nuclear transcription factors CAR, PXR and AHR, and in which the endogenous equivalent genes have been rendered inoperable.

23. An animal according to claim 21, which has been additionally humanised for the AHR receptor, and in which the endogenous equivalent gene has been rendered inoperable.

24. An animal according to claim 21, in which all endogenous equivalent genes have been rendered inoperable in all tissues.

25. An animal according to claim 21, in which the expression level of the genes rendered inoperable is less than 10% of the wild type expression level.

26. An animal according to claim 21, wherein the human transcription factor gene sequences are inserted at the point in the host animal chromosome where the endogenous equivalent target genes naturally occur.

27. An animal according to claim 21, wherein the insertion of the human transcription factor genes at the point in the host animal chromosome where the endogenous equivalent target genes naturally occur replaces the endogenous equivalent target genes in the host chromosomes.

28. An animal according to claim 21, wherein transcription of said human transcription factor genes is under the control of one or more endogenous regulatory sequence(s) of the host animal.

29. An animal according to claim 21, wherein transcription of said human replacement gene sequence is under the control of the endogenous human regulatory sequence(s).

30. An animal according to claim 21, which has additionally been humanised for one, two or all three of CYP3A4, CYP2C9, and CYP2D6.

31. An animal according to claim 21, which has additionally been humanised for MDR and/or MRP.

32. An animal according to claim 21, which has additionally been humanised for UGT1A.

33. An animal according to claim 21, which is a rodent, more preferably, a mouse.

34. A cell isolated from an animal according to claim 21.

35. A cell according to claim 34 which is a stem cell.

36. A cell according to claim 35 which is an embryonic stem cell.
